(19) United States
(12) Patent Application Publication
(10) Pub. No.: US 2012/0225952 A1
(43) Pub. Date: Sep. 6, 2012

(54) NON-AQUEOUS SILICONE-BASED OPHTHALMIC FORMULATIONS

(76) Inventors: Kevin S. Warner, Anaheim, CA (US); Ajay Parashar, Irvine, CA (US)

(21) Appl. No.: 13/411,059

(22) Filed: Mar. 2, 2012

Related U.S. Application Data
(60) Provisional application No. 61/448,899, filed on Mar. 3, 2011.
(60) Provisional application No. 61/529,553, filed on Aug. 31, 2011.
(60) Provisional application No. 61/565,447, filed on Nov. 30, 2011.
(60) Provisional application No. 61/448,890, filed on Mar. 3, 2011.

(51) Int. Cl. A61K 47/24; A61K 47/34; A61P 27/02

(52) U.S. Cl. 514/772.3; 514/772

(57) ABSTRACT

Non-aqueous compositions including a silicone based excipient and methods of their use are provided. The non-aqueous compositions, products and methods of the present invention are particularly useful for the treatment of ophthalmic diseases

### CROSS-REFERENCE TO RELATED APPLICATION

This application is based, and claims priority under 35 U.S.C. §120 to U.S. Provisional Patent Application Nos. 61/448,899 filed on Mar. 3, 2011, 61/529,553 filed on Aug. 31, 2011, 61/565,447 filed on Nov. 30, 2011, and 61/448,890 filed on Mar. 3, 2011, each of which is incorporated herein by reference.

### BACKGROUND OF THE INVENTION

The eye can be inflicted with diseases and conditions which require specialized medical treatments (See Afshari N., Research in cornea and external disease in refining current concept and branching out into new avenues of investigation, Rev Ophthalmol Online, April 2006, 5 (13); and Schroeder, I., et al., Development and characterization of film forming polymeric solutions for skin drug delivery, European Journal of Pharmaceutics and Biopharmaceutics, January 2007, 65 (1), p. 111-121). In order to effectively deliver a pharmaceutically active composition to the eye, appropriate vehicles are required. There is a need in the field for effective ophthalmic vehicles (e.g. excipients), which are chemically and biologically inert, have a low surface tension (e.g. good spreading characteristics on water), enable the solubility of hydrophobic drugs and maintain drug efficacy without side effects. The present invention solves these as well as other problems in the art by, inter alia providing non-aqueous silicone based topical ophthalmic formulations for application to the region on and around the eye (i.e. conjunctiva, lacrima tissue or cornea) and maintaining efficacy without side effects.

### BRIEF SUMMARY OF THE INVENTION

Presented herein inter alia are non-aqueous compositions containing silicone based excipients for ophthalmic application as well as methods of treating ophthalmic diseases and methods of improving vision. In certain embodiments, the non-aqueous compositions and methods are useful for treating the symptoms of glaucoma and include a combination of active pharmaceutical ingredients and a silicone excipient.

In one aspect, a non-aqueous composition including an active pharmaceutical ingredient and a silicone excipient is provided.

In another aspect, a method of treating an ophthalmic disease in a subject in need thereof is provided. The method includes administering to the subject an active pharmaceutical ingredient and a silicone excipient.

In another aspect, a method of improving vision in a subject in need thereof is provided. The method includes administering to the subject an active pharmaceutical ingredient and a silicone excipient.

Some embodiments of the invention include the following:

Embodiment 1. A non-aqueous composition comprising an active pharmaceutical ingredient and a silicone excipient.

Embodiment 2. The non-aqueous composition of embodiment 1, wherein said active pharmaceutical ingredient is an immunosuppressant, a vasodilator agent, an anti-inflammatory agent, an EP2 receptor agonist, a muscarinic receptor agonist, a prostaglandin analog, a vasoconstrictor agent, or an anti-infective agent.

Embodiment 3. The non-aqueous composition of embodiment 1, wherein said composition is an ophthalmic pharmaceutical formulation.

Embodiment 4. The non-aqueous composition of embodiment 1, wherein said active pharmaceutical ingredient is an immunosuppressant

Embodiment 5. The non-aqueous composition of embodiment 4, wherein said immunosuppressant is cyclosporine.

Embodiment 6. The non-aqueous composition of embodiment 5, wherein said cyclosporine is present in an amount approximately equal to or less than about 0.1% w/w.

Embodiment 7. The non-aqueous composition of embodiment 5, wherein said cyclosporine is present in an amount of about 0.01% w/w

Embodiment 8. The non-aqueous composition of embodiment 4, wherein said immunosuppressant is tacrolimus.

Embodiment 9. The non-aqueous composition of embodiment 8, wherein said tacrolimus is present in an amount approximately equal to or less than about 4% w/w.

Embodiment 10. The non-aqueous composition of embodiment 8, wherein said tacrolimus is present in an amount of about 0.001% w/w.

Embodiment 11. The non-aqueous composition of embodiment 1, wherein said active pharmaceutical ingredient is a vasodilator agent.

Embodiment 12. The non-aqueous composition of embodiment 11, wherein said vasodilator agent is an alpha adrenergic antagonist

Embodiment 13. The non-aqueous composition of embodiment 12, wherein said alpha adrenergic antagonist is phentolamine.

Embodiment 14. The non-aqueous composition of embodiment 12, wherein said phentolamine is present in an amount approximately equal to or less than about 4% w/w

Embodiment 15. The non-aqueous composition of embodiment 12, wherein said phentolamine is present in an amount of about 0.001% w/w

Embodiment 16. The non-aqueous composition of embodiment 1, wherein said active pharmaceutical ingredient is an anti-inflammatory agent.

Embodiment 17. The non-aqueous composition of embodiment 16, wherein said anti-inflammatory agent is a non-steroidal anti-inflammatory agent.

Embodiment 18. The non-aqueous composition of embodiment 17, wherein said non-steroidal anti-inflammatory agent is ketorolac.

Embodiment 19. The non-aqueous composition of embodiment 18, wherein said ketorolac is present in an amount approximately equal to or less than about 2% w/w.

Embodiment 20. The non-aqueous composition of embodiment 18, wherein said ketorolac is present in an amount of about 0.01% w/w.

Embodiment 21. The non-aqueous composition of embodiment 16, wherein said anti-inflammatory agent is testosterone.

Embodiment 22. The non-aqueous composition of embodiment 21, wherein said testosterone is present in an amount approximately equal to or less than about 5% w/w.

Embodiment 23. The non-aqueous composition of embodiment 21, wherein said testosterone is present in an amount of about 0.001% w/w.

Embodiment 24. The non-aqueous composition of embodiment 16, wherein said anti-inflammatory agent is dihydrotestosterone.

Embodiment 25. The non-aqueous composition of embodiment 24, wherein said dihydrotestosterone is present in an amount approximately equal to or less than about 5% w/w.

Embodiment 26. The non-aqueous composition of embodiment 24, wherein said dihydrotestosterone is present in an amount of about 0.001% w/w.

Embodiment 27. The non-aqueous composition of embodiment 16, wherein said anti-inflammatory agent is testosterone propionate.

Embodiment 28. The non-aqueous composition of embodiment 27, wherein said testosterone propionate is present in an amount approximately equal to or less than about 5% w/w.

Embodiment 29. The non-aqueous composition of embodiment 27, wherein said testosterone propionate is present in an amount of about 0.001% w/w.

Embodiment 30. The non-aqueous composition of embodiment 16, wherein said anti-inflammatory agent is dexamethasone.

Embodiment 31. The non-aqueous composition of embodiment 30, wherein said dexamethasone is present in amount approximately equal to or less than about 5% w/w.

Embodiment 32. The non-aqueous composition of embodiment 30, wherein said dexamethasone is present in an amount of about 0.001% w/w.

Embodiment 33. The non-aqueous composition of embodiment 16, wherein said anti-inflammatory agent is prednisolone.

Embodiment 34. The non-aqueous composition of embodiment 33, wherein said prednisolone is present in amount approximately equal to or less than about 5% w/w.

Embodiment 35. The non-aqueous composition of embodiment 33, wherein said prednisolone is present in amount of about 0.001% w/w.

Embodiment 36. The non-aqueous composition of embodiment 1, wherein said active pharmaceutical ingredient is an EP2 receptor agonist.

Embodiment 37. The non-aqueous composition of embodiment 36, wherein said EP2 receptor agonist has the formula

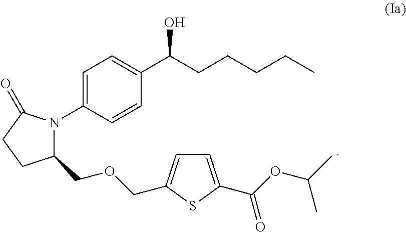

Embodiment 38. The non-aqueous composition of embodiment 37, wherein said EP2 receptor agonist is present in an amount approximately equal to or less than about 0.1% w/w.

Embodiment 39. The non-aqueous composition of embodiment 37, wherein said EP2 receptor agonist is present in an amount of about 0.001% w/w.

Embodiment 40. The non-aqueous composition of embodiment 36, wherein said EP2 receptor agonist has the formula

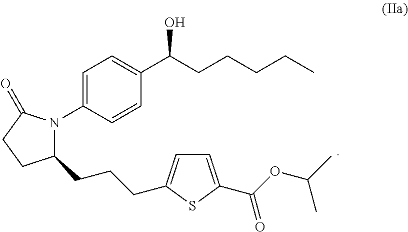

Embodiment 41. The non-aqueous composition of embodiment 40, wherein said EP2 receptor agonist is present in an amount approximately equal to or less than about 0.05% w/w.

Embodiment 42. The non-aqueous composition of embodiment 40, wherein said EP2 receptor agonist is present in an amount of about 0.0002% w/w.

Embodiment 43. The non-aqueous composition of embodiment 36, wherein said EP2 receptor agonist has the formula

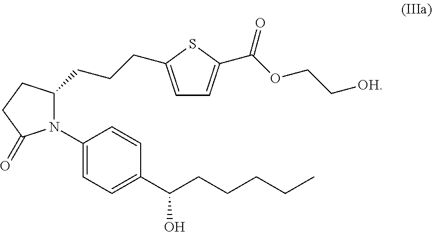

Embodiment 44. The non-aqueous composition of embodiment 43, wherein EP2 receptor agonist is present in an amount approximately equal to or less than about 0.1% w/w.

Embodiment 45. The non-aqueous composition of embodiment 43, wherein said EP2 receptor agonist is present in an amount of about 0.001% w/w.

Embodiment 46. The non-aqueous composition of embodiment 1, wherein said active pharmaceutical ingredient is a muscarinic receptor agonist.

Embodiment 47. The non-aqueous composition of embodiment 46, wherein said muscarinic receptor agonist is pilocarpine.

Embodiment 48. The non-aqueous composition of embodiment 47, wherein said pilocarpine is present in an amount approximately equal to or less than about 6% w/w.

Embodiment 49. The non-aqueous composition of embodiment 47, wherein said pilocarpine is present in an amount of about 0.1% w/w.

Embodiment 50. The non-aqueous composition of embodiment 1, wherein said active pharmaceutical ingredient is a prostaglandin analog.

Embodiment 51. The non-aqueous composition of embodiment 50, wherein said prostaglandin analog is bimatoprost.

Embodiment 52. The non-aqueous composition of embodiment 51, wherein said bimatoprost is present in an amount approximately equal to or less than about 0.1% w/w.

Embodiment 53. The non-aqueous composition of embodiment 51, wherein said bimatoprost is present in an amount of about 0.001% w/w.

Embodiment 54. The non-aqueous composition of embodiment 50, wherein said prostaglandin analog is latanoprost.

Embodiment 55. The non-aqueous composition of embodiment 54, wherein said latanoprost is present in an amount approximately equal to or less than about 0.1% w/w.

Embodiment 56. The non-aqueous composition of embodiment 54, wherein said latanoprost is present in an amount of about 0.0003% w/w.

Embodiment 57. The non-aqueous composition of embodiment 50, wherein said prostaglandin analog is travoprost.

Embodiment 58. The non-aqueous composition of embodiment 57, wherein said travoprost is present in an amount approximately equal to or less than about 0.1% w/w.

Embodiment 59. The non-aqueous composition of embodiment 57, wherein said travoprost is present in an amount of about 0.0002% w/w.

Embodiment 60. The non-aqueous composition of embodiment 1, wherein said active pharmaceutical ingredient is a vasoconstrictor agent.

Embodiment 61. The non-aqueous composition of embodiment 60, wherein said vasoconstrictor agent is an alpha adrenergic agonist.

Embodiment 62. The non-aqueous composition of embodiment 61, wherein said alpha adrenergic agonist is brimonidine.

Embodiment 63. The non-aqueous composition of embodiment 62, wherein said brimonidine is present in an amount approximately equal to or less than 1% w/w.

Embodiment 64. The non-aqueous composition of embodiment 62, wherein said brimonidine is present in an amount of about 0.001% w/w.

Embodiment 65. The non-aqueous composition of embodiment 61, wherein said alpha adrenergic agonist is an alpha adrenergic agonist compound.

Embodiment 66. The non-aqueous composition of embodiment 65, wherein said alpha adrenergic agonist compound has the Formula

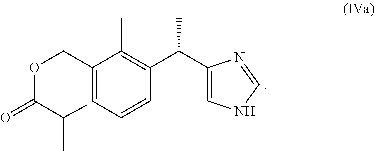

Embodiment 67. The non-aqueous composition of embodiment 65, wherein said alpha adrenergic agonist compound has the Formula

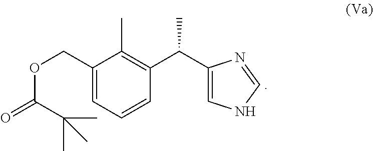

Embodiment 68. The non-aqueous composition of embodiment 65, wherein said alpha adrenergic agonist compound has the Formula

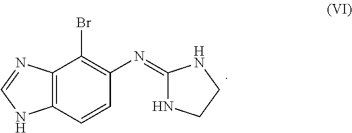

Embodiment 69. The non-aqueous composition of embodiment 65, wherein said alpha adrenergic agonist compound has the Formula

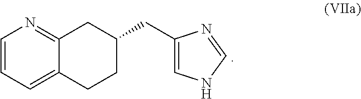

Embodiment 70. The non-aqueous composition of embodiment 65, wherein said alpha adrenergic agonist compound has the Formula

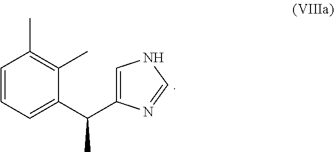

Embodiment 71. The non-aqueous composition of embodiment 65, wherein said alpha adrenergic agonist compound is present in an amount approximately equal to or less than 1% w/w.

Embodiment 72. The non-aqueous composition of embodiment 65, wherein said alpha adrenergic agonist compound is present in an amount of about 0.001% w/w.

Embodiment 73. The non-aqueous composition of embodiment 60, wherein said vasoconstrictor agent is a beta adrenergic antagonist.

Embodiment 74. The non-aqueous composition of embodiment 73, wherein said beta adrenergic antagonist is timolol.

Embodiment 75. The non-aqueous composition of embodiment 74, wherein said timolol is present in an amount approximately equal to or less than about 0.5% w/w.

Embodiment 76. The non-aqueous composition of embodiment 74, wherein said timolol is present in amount of about 0.05% w/w.

Embodiment 77. The non-aqueous composition of embodiment 1, wherein said active pharmaceutical ingredient is an anti-infective agent.

Embodiment 78. The non-aqueous composition of embodiment 77, wherein said anti-infective agent is gatifloxacin.

Embodiment 79. The non-aqueous composition of embodiment 78, wherein said gatifloxacin is present in an amount approximately equal to or less than about 1% w/w.

Embodiment 80. The non-aqueous composition of embodiment 78, wherein said gatifloxacin is present in an amount of about 0.1% w/w.

Embodiment 81. The non-aqueous composition of embodiment 1, wherein said silicone excipient is a first silicone excipient blend, a second silicone excipient blend, a third silicone excipient blend, fourth silicone excipient blend, a fifth silicone excipient blend, a sixth silicone excipient blend or a seventh silicone excipient blend.

Embodiment 82. The non-aqueous composition of embodiment 81, wherein said composition comprises a first silicone excipient blend and a second silicone excipient blend.

Embodiment 83. The non-aqueous composition of embodiment 81, wherein said composition comprises a first silicone excipient blend, a second silicone excipient blend and a third silicone excipient blend.

Embodiment 84. The non-aqueous composition of embodiment 81, wherein said composition comprises a first silicone excipient blend, a second silicone excipient blend, a third silicone excipient blend and a fourth silicone excipient blend.

Embodiment 85. The non-aqueous composition of embodiment 81, wherein said first silicone excipient blend comprises a mixture of dimethicone and dimethiconol.

Embodiment 86. The non-aqueous composition of embodiment 85, wherein said first silicone excipient blend is present from about 1% w/w to about 10% w/w.

Embodiment 87. The non-aqueous composition of embodiment 81, wherein said second silicone excipient blend comprises a mixture of cyclopentasiloxane and a dimethicone cross polymer.

Embodiment 88. The non-aqueous composition of embodiment 88, wherein said second silicone excipient blend is present from about 5% w/w to about 20% w/w.

Embodiment 89. The non-aqueous composition of embodiment 81, wherein said third silicone excipient blend comprises a mixture of polydimethylcyclosiloxanes.

Embodiment 90. The non-aqueous composition of embodiment 89, wherein said third silicone excipient blend is present from about 10% w/w to about 30% w/w.

Embodiment 91. The non-aqueous composition of embodiment 81, wherein said fourth silicone excipient blend comprises a mixture of alkylmethyl siloxane copolyol, isostearyl alcohol and 1-dodecene.

Embodiment 92. The non-aqueous composition of embodiment 91, wherein said fourth silicone excipient blend is present from about 0.5% w/w to about 5% w/w.

Embodiment 93. The non-aqueous composition of embodiment 81, wherein said fifth silicone excipient blend comprises a mixture of stearyloxytrimethylsilane and stearyl alcohol.

Embodiment 94. The non-aqueous composition of embodiment 93, wherein said fifth silicone excipient blend is present from about 5% w/w to about 15% w/w.

Embodiment 95. The non-aqueous composition of embodiment 81, wherein said sixth silicone excipient blend comprises a mixture of dimethiconol and hexamethyldisiloxane.

Embodiment 96. The non-aqueous composition of embodiment 95, wherein said sixth silicone excipient blend is present from about 5% w/w to about 10% w/w.

Embodiment 97. The non-aqueous composition of embodiment 81, wherein said seventh silicone excipient blend comprises alkylmethyl siloxane wax.

Embodiment 98. The non-aqueous composition of embodiment 97, wherein said seventh silicone excipient blend is present from about 5% w/w to about 12% w/w.

Embodiment 99. The non-aqueous composition of embodiment 1, further comprising a plurality of lipid excipients or a thickening agent.

Embodiment 100. The non-aqueous composition of embodiment 1, further comprising a plurality of lipid excipients and a thickening agent.

Embodiment 101. The non-aqueous composition of embodiment 99, wherein said thickening agent is talc.

Embodiment 102. The non-aqueous composition embodiment 101, wherein said talc is present from about 2% w/w to about 5% w/w.

Embodiment 103. The non-aqueous composition of embodiment 1, wherein said active pharmaceutical ingredient is selected from the group consisting of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, and gatifloxacin.

Embodiment 104. The non-aqueous composition of embodiment 103, wherein cyclosporine is present from 0.01% w/w about to about 0.1% w/w.

Embodiment 105. The non-aqueous composition of embodiment 103, wherein tacrolimus is present from about 0.01% w/w to about 0.1% w/w.

Embodiment 106. The non-aqueous composition of embodiment 103, wherein phentolamine is present from about 0.0001% w/w to about 1% w/w.

Embodiment 107. The non-aqueous composition of embodiment 103, wherein testosterone is present from about 0.001% w/w to about 5% w/w.

Embodiment 108. The non-aqueous composition of embodiment 103, wherein dihydrotestosteron is present from about 0.001% w/w to about 5% w/w.

Embodiment 109. The non-aqueous composition of embodiment 103, wherein testosterone propionate is present from about 0.001% w/w to about 5% w/w.

Embodiment 110. The non-aqueous composition of embodiment 103, wherein said EP2 receptor agonist has the Formula

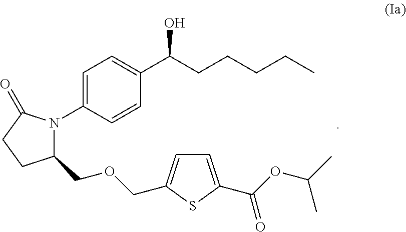

Embodiment 111. The non-aqueous composition of embodiment 110, wherein said EP2 receptor agonist is present from about 0.001% w/w to about 0.1% w/w.

Embodiment 112. The non-aqueous composition of embodiment 103, wherein said

EP2 receptor agonist has the Formula

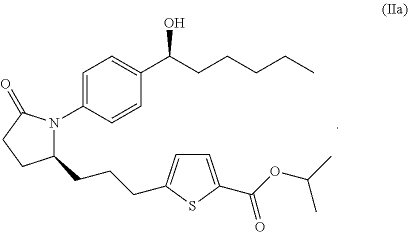

Embodiment 113. The non-aqueous composition of embodiment 112, wherein said EP2 receptor agonist is present from about 0.0002% w/w to about 0.05% w/w.

Embodiment 114. The non-aqueous composition of embodiment 1, consisting essentially of: an active pharmaceutical ingredient selected from the group consisting of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, and gatifloxacin; a plurality of lipid excipients; and one or more silicone excipients.

Embodiment 115. The non-aqueous composition of embodiment 1, consisting essentially of: an active pharmaceutical ingredient selected from the group consisting of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, and gatifloxacin; a plurality of lipid excipients; a thickening agent; and one or more silicone excipients.

Embodiment 116. The non-aqueous composition of embodiment 3, wherein said ophthalmic pharmaceutical formulation is an ointment formulation.

Embodiment 117. The non-aqueous composition of embodiment 116, wherein said active pharmaceutical ingredient is selected from the group consisting of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, and gatifloxacin.

Embodiment 118. The non-aqueous composition of embodiment 117, wherein said silicone excipient is a first silicone blend or a second silicone blend.

Embodiment 119. The non-aqueous composition of embodiment 118 comprising a first silicone excipient blend and a second silicone excipient blend.

Embodiment 120. The non-aqueous composition of embodiment 119, wherein said first silicone excipient blend is a mixture of dimethicone and dimethiconol and said second silicone excipient blend is a mixture of alkylmethyl siloxane wax.

Embodiment 121. The non-aqueous composition of embodiment 119, wherein said first silicone excipient blend is a mixture of cyclopentasiloxane and dimethicone cross polymer and said second silicone excipient blend is a mixture of polydimethylcyclosiloxanes.

Embodiment 122. The non-aqueous composition of embodiment 119, further comprising a lipid excipient.

Embodiment 123. The non-aqueous composition of embodiment 3, wherein said ophthalmic pharmaceutical formulation is a gel formulation.

Embodiment 124. The non-aqueous composition of embodiment 123, wherein said active pharmaceutical ingredient is selected from the group consisting of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, and gatifloxacin.

Embodiment 125. The non-aqueous composition of claim embodiment 124, wherein said silicone excipient is a first silicone excipient blend or a second silicone excipient blend.

Embodiment 126. The non-aqueous composition of embodiment 125 comprising a first silicone excipient blend and a second silicone excipient blend.

Embodiment 127. The non-aqueous composition of embodiment 126, wherein said first silicone excipient blend is a mixture of cyclopentasiloxane and dimethicone cross polymer and said second silicone excipient blend is a mixture of polydimethylcyclosiloxanes.

Embodiment 128. The non-aqueous composition of embodiment 126, further comprising a lipid excipient.

Embodiment 129. The non-aqueous composition of embodiment 3 wherein said ophthalmic pharmaceutical formulation is a spray formulation.

Embodiment 130. The non-aqueous composition of embodiment 129, wherein said active pharmaceutical ingredient is selected from the group consisting of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, and gatifloxacin.

Embodiment 131. The non-aqueous composition of embodiment 130, wherein said silicone excipient is a silicone excipient blend, said silicone excipient blend comprising a mixture of dimethiconol and hexamethyldisiloxane.

Embodiment 132. The non-aqueous composition of embodiment 131, further comprising a thickening agent.

Embodiment 133. The non-aqueous composition of embodiment 130, wherein said silicone excipient is a first silicone excipient blend or a second silicone excipient blend.

Embodiment 134. The non-aqueous composition of embodiment 130 comprising a first silicone excipient blend and a second silicone excipient blend.

Embodiment 135. The non-aqueous composition of embodiment 134, wherein said first silicone excipient blend is a mixture of cyclopentasiloxane and dimethicone cross polymer and said second silicone excipient blend is a mixture of dimethiconol and hexamethyldisiloxane.

Embodiment 136. The non-aqueous composition of embodiment 130, wherein said silicone excipient is a first silicone excipient blend, a second silicone excipient blend or a third silicone excipient blend.

Embodiment 137. The non-aqueous composition of embodiment 136 comprising a first silicone excipient blend, a second silicone excipient blend and a third silicone excipient blend.

Embodiment 138. The non-aqueous composition of embodiment 137, wherein said first silicone excipient blend is a mixture of dimethicone and dimethiconol, said second silicone excipient blend is a mixture of cyclopentasiloxane and dimethicone cross polymer, and said third silicone excipient blend is a mixture of polydimethylcyclosiloxanes.

Embodiment 139. The non-aqueous composition of embodiment 3, wherein said ophthalmic pharmaceutical formulation is a stick formulation.

Embodiment 140. The non-aqueous composition of embodiment 139, wherein said active pharmaceutical ingredient is selected from the group consisting of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, and gatifloxacin.

Embodiment 141. The non-aqueous composition of embodiment 140, wherein said silicone excipient is an alkylmethyl siloxane wax.

Embodiment 142. The non-aqueous composition of embodiment 141, further comprising a plurality of lipid excipients.

Embodiment 143. The non-aqueous composition of embodiment 140, wherein said silicone excipient is a first silicone excipient blend or a second silicone excipient blend.

Embodiment 144. The non-aqueous composition of embodiment 143, comprising a first silicone excipient blend and a second silicone excipient blend.

Embodiment 145. The non-aqueous composition of embodiment 144, wherein said first silicone excipient blend is a mixture of stearyloxytrimethylsilane and stearyl alcohol, and said second silicone excipient blend is a mixture of polydimethylcyclosiloxanes.

Embodiment 146. The non-aqueous composition of embodiment 145, further comprising a plurality of lipid excipients.

Embodiment 147. The non-aqueous composition of embodiment 3 wherein said ophthalmic pharmaceutical formulation is an emulsion formulation.

Embodiment 148. The non-aqueous composition of embodiment 147 wherein said active pharmaceutical ingredient is selected from the group consisting of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, and gatifloxacin.

Embodiment 149. The non-aqueous composition of embodiment 148 wherein said silicone excipient is a mixture of alkylmethyl siloxane copolyol, isostearyl alcohol and 1-dodecene.

Embodiment 150. The non-aqueous composition of embodiment 149 further comprising a lipid excipient.

Embodiment 151. The non-aqueous composition of embodiment 148 wherein said silicone excipient is a first silicone excipient blend or a second silicone excipient blend.

Embodiment 152. The non-aqueous composition of embodiment 151 comprising a first silicone excipient blend and a second silicone excipient blend.

Embodiment 153. The non-aqueous composition of embodiment 152, wherein said first silicone excipient blend is a mixture of alkylmethyl siloxane copolyol, isostearyl alcohol and 1-dodecene, and said second silicone excipient blend is a mixture of dimethicone and dimethiconol.

Embodiment 154. The non-aqueous composition of embodiment 153, further comprising a lipid excipient.

Embodiment 155. A method of treating an ophthalmic disease in a subject in need thereof, said method comprising administering to said subject an active pharmaceutical ingredient and a silicone excipient.

Embodiment 156. The method of embodiment 155, wherein said ophthalmic disease is central retinal vein occlusion.

Embodiment 157. The method of embodiment 155, wherein said ophthalmic disease is branch retinal vein occlusion.

Embodiment 158. The method of embodiment 155, wherein said ophthalmic disease is choroidal macular edema.

Embodiment 159. The method of embodiment 155, wherein said ophthalmic disease is diabetic macular edema.

Embodiment 160. The method of embodiment 155, wherein said ophthalmic disease is diabetic macular retinopathy.

Embodiment 161. The method of embodiment 155, wherein said ophthalmic disease is uveitis.

Embodiment 162. The method of embodiment 155, wherein said ophthalmic disease is age related macular degeneration.

Embodiment 163. The method of embodiment 155, wherein said ophthalmic disease is glaucoma.

Embodiment 164. The method of embodiment 155, wherein said ophthalmic disease is ocular hypertension.

Embodiment 165. A method of improving vision in a subject in need thereof, said method comprising administering to said subject an active pharmaceutical ingredient and a silicone excipient.

### BRIEF DESCRIPTION OF THE DRAWINGS

FIG. 1. Lowering intra-ocular pressure (TOP) in normotensive rabbits as a function of time.

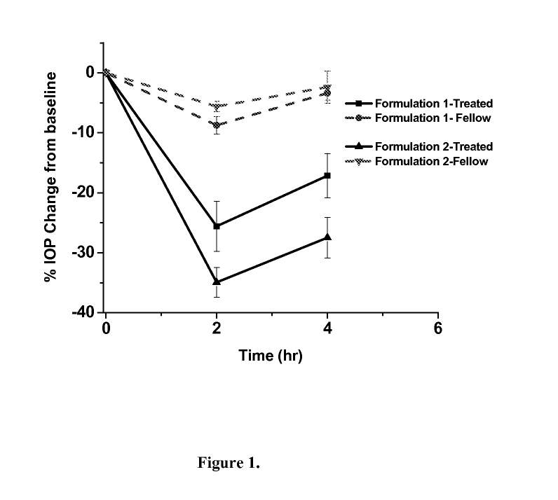

### DETAILED DESCRIPTION OF THE INVENTION

### I. Definitions

The terms “a,” “an,” or “the” as used herein not only include aspects with one member, but also aspects with more than one member. For example, an embodiment including “a buffer and a chelating agent” should be understood to present aspects with at least a second buffer, at least a second chelating agent, or both.

The term “or” as used herein should in general be construed non-exclusively. For example, an embodiment of “a formulation including A or B” would typically present an aspect with a formulation including both A and B. “Or” should, however, be construed to exclude those aspects presented that cannot be combined without contradiction (e.g., a formulation pH that is between 9 and 10 or between 7 and 8).

“Agent” as used herein indicates a compound or mixture of compounds that, when added to a pharmaceutical formulation, tend to produce a particular effect on the formulation's properties. For example, a formulation including a thickening agent is likely to be more viscous than an otherwise identical comparative formulation that lacks the thickening agent.

“Formulation,” “composition,” and “preparation” as used herein are equivalent terms referring to a composition of matter suitable for pharmaceutical use (i.e., producing a therapeutic effect as well as possessing acceptable pharmacokinetic and toxicological properties).

The term “non-aqueous” composition or formulation (e.g. non-aqueous ophthalmic compositions) as provided herein refers to a composition where water is present at an amount approximately equal to or less than 20% w/w. In some embodiments, water is present at an amount less than 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, 0.1, 0.01, 0.001, 0.0001, 0.00001, or 0.000001% w/w. In some embodiments, water is present at an amount less than 5, 4, 3, 2, 1, 0.9, 0.8, 0.7, 0.6, 0.5, 0.4, 0.3, 0.2, 0.1, 0.01, 0.001, 0.0001, 0.00001, or 0.000001% w/w. In some embodiments, water is present at an amount less than 1, 0.9, 0.8, 0.7, 0.6, 0.5, 0.4, 0.3, 0.2, 0.1, 0.01, 0.001, 0.0001, 0.00001, or 0.000001% w/w. In some embodiments, water is present at an amount less than 0.5, 0.4, 0.3, 0.2, 0.1, 0.01, 0.001, 0.0001, 0.00001, or 0.000001% w/w. In some embodiments, water is present at an amount less than 1% w/w. In some embodiments, water is present at an amount less than 0.5% w/w. In some embodiments, water is present at an amount less than 0.1% w/w. In some embodiments, water is present at an amount less than 0.01% w/w. In some embodiments, water is present at an amount less than 0.001% w/w. In some embodiments, water is present at an amount less than 0.0001% w/w. In some embodiments, water is present at an amount less than 0.00001% w/w. In some embodiments, water is present at an amount less than 0.000001% w/w. In some embodiments, water is present at an amount less than 0.0000001% w/w. In some embodiments, water is present in trace amounts. In some embodiments, water is absent. In other embodiments, the non-aqueous composition includes traces of water. In other embodiments, the non-aqueous composition includes no water.

As used herein, the term “pharmaceutically” acceptable is used as equivalent to physiologically acceptable. In certain embodiments, a pharmaceutically acceptable composition or preparation will include agents for buffering and preservation in storage, and can include buffers and carriers for appropriate delivery, depending on the route of administration.

As used herein, the terms “prevent” and “treat” are not intended to be absolute terms. Treatment can refer to any delay in onset, e.g., reduction in the frequency or severity of symptoms, amelioration of symptoms, improvement in patient comfort, reduction in skin inflammation, and the like. The effect of treatment can be compared to an individual or pool of individuals not receiving a given treatment, or to the same patient before, or after cessation of, treatment.

The terms “subject,” “patient,” “individual,” and the like as used herein are not intended to be limiting and can be generally interchanged. That is, an individual described as a “patient” does not necessarily have a given disease, but may be merely seeking medical advice.

The term “subject” as used herein includes all members of the animal kingdom prone to suffering from the indicated disorder. In some aspects, the subject is a mammal, and in some aspects, the subject is a human.

The terms “effective amount,” “therapeutically effective amount” or “pharmaceutically effective amount” as used herein refers to that amount of the therapeutic agent sufficient to ameliorate one or more aspects of the disorder. The result can be reduction and/or alleviation of the signs, symptoms, or causes of a disease, or any other desired alteration of a biological system. For example, an “effective amount” for therapeutic uses is the amount of the composition comprising an agent as set forth herein required to provide a clinically significant decrease in an ophthalmic disease. For example, for the given aspect (e.g., length of incidence), a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. An appropriate “effective” amount in any individual case may be determined using techniques, such as a dose escalation study.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “disease” refers to any deviation from the normal health of a mammal and includes a state when disease symptoms are present, as well as conditions in which a deviation (e.g., infection, gene mutation, genetic defect, etc.) has occurred, but symptoms are not yet manifested. According to the present invention, the methods disclosed herein are suitable for use in a patient that is a member of the Vertebrate class, Mammalia, including, without limitation, primates, livestock and domestic pets (e.g., a companion animal). Typically, a patient will be a human patient.

As used herein, “topical application,” “topical administration,” and “topically administering” are used interchangeably herein and include the administration of a composition to the eye, the mucosal or dermal area proximal to the eye. Topical application or administering may result in the delivery of an active agent to the eye or skin, a localized region of the body, a localized volume of the body, or the systemic circulation.

“Topical formulation” and “topical pharmaceutical composition” are used interchangeably herein and include a formulation that is suitable for topical application to the eye or dermal area proximal to the eye, or other localized region of the body. A topical formulation may, for example, be used to confer a therapeutic benefit to its user. Specific topical formulations can be used for topical, local, regional, or transdermal application of substances.

As used herein, the terms “application,” “apply,” and “applying” used in reference to a topical composition product or method of using a composition or a product, refer to any manner of administering a topical composition or a product to the eye, the mucosal or dermal area proximal to the eye of a patient which, in medical or cosmetology practice, delivers the composition or the product to patient's eye, the mucosal or dermal area proximal to the eye. Smearing, rubbing, spreading, spraying a topical composition, with or without the aid of suitable devices, on a patient's skin are all included within the scope of the term “application,” as used herein. The term “topical” or “topically” in reference to administration or application of a composition or a product refers to epicuatenous administration or application, or administration onto skin. The term “topically active agent” as used herein refers to a compound that is effective in a treatment of a skin condition when administered topically. It is to be understood that topically active agent can have a local or a systemic effect, or both, when administered topically. The term “topical,” when used in reference to a composition or a product refers to a composition or a product formulated for topical application.

The abbreviations used herein have their conventional meaning within the chemical, biological or pharmaceutical arts.

The terms “about” and “approximately equal” are used herein to modify a numerical value and indicate a defined range around that value. If “X” were the value, “about X” or “approximately equal to X” would generally indicate a value from 0.90X to 1.10X. Any reference to “about X” minimally indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. Thus, “about X” is intended to disclose, e.g., “0.98X.” When “about” is applied to the beginning of a numerical range, it applies to both ends of the range. Thus, “from about 6 to 8.5” is equivalent to “from about 6 to about 8.5.” When “about” is applied to the first value of a set of values, it applies to all values in that set. Thus, “about 7, 9, or 11%” is equivalent to “about 7%, about 9%, or about 11%.”

As used herein, the phrase “pharmaceutically acceptable salts” refers to salts of the active compound(s) which possess the same pharmacological activity as the active compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, naturally and synthetically derived amino acids. Non-limiting examples of base salts include ammonium salts; alkali metal salts, such as sodium and potassium salts; alkaline earth metal salts, such as calcium and magnesium salts; salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others.

In formulations including an “additional,” “further,” or “second” component, the second component as used herein is chemically different from the other components or first component. A “third” component is different from the other, first, and second components, and further enumerated or “additional” components are similarly different.

The term “hydrophobic” is used herein in accordance with its plain ordinary meaning and refers to a chemical group having a tendency to attract non-polar or uncharged chemical groups, e.g. hexane, and to repel polar or charged chemical groups, e.g. water.

The term “hydrophilic” is used herein in accordance with its plain ordinary meaning and refers to a chemical group having a tendency to repel non-polar or uncharged chemical groups, e.g. hexane, and to attract polar or charged chemical groups, e.g. water.

### II. Compositions

The present invention provides non-aqueous pharmaceutical compositions including a pharmaceutically active ingredient (e.g. multiple pharmaceutically active ingredients) and a silicone excipient. In some embodiments, the silicone excipient is a silicone excipient blend. The non-aqueous pharmaceutical composition may have multiple silicone excipient blends. The silicone based non-aqueous pharmaceutical compositions provided herein may be used for the treatment of ophthalmic diseases. Ointments, gels, sprays, stick formulations and emulsions are contemplated as useful pharmaceutical formulations including the compositions provided herein.

In one aspect, a non-aqueous composition including an active pharmaceutical ingredient (also referred to herein as an “active ingredient”) and a silicone excipient is provided. In some embodiments, the non-aqueous composition is an ophthalmic pharmaceutical formulation (i.e. a pharmaceutical formulation suitable for use ophthalmically and having ophthalmically acceptable excipients). The active pharmaceutical ingredients are present in an amount effective to treat ophthalmic diseases.

The non-aqueous compositions provided herein may include an immunosuppressant, a vasodilator agent, an anti-inflammatory agent, an EP2 receptor agonist, a muscarinic receptor agonist, a prostaglandin analog, a vasoconstrictor agent, or an anti-infective agent as active pharmaceutical ingredients. In some embodiments, the non-aqueous composition provided herein includes an immunosuppressant (e.g. in the absence of another active ingredient). In some embodiments, the non-aqueous composition provided herein includes an vasodilator agent (e.g. in the absence of another active ingredient). In some embodiments, the non-aqueous composition provided herein includes an anti-inflammatory agent (e.g. in the absence of another active ingredient). In some embodiments, the non-aqueous composition provided herein includes an EP2 receptor agonist (e.g. in the absence of another active ingredient). In some embodiments, the non-aqueous composition provided herein includes a muscarinic receptor agonist (e.g. in the absence of another active ingredient). In some embodiments, the non-aqueous composition provided herein includes a prostaglandin analog (e.g. in the absence of another active ingredient). In some embodiments, the non-aqueous composition provided herein includes a vasoconstrictor agent (e.g. in the absence of another active ingredient). In some embodiments, the non-aqueous composition provided herein includes an anti-infective agent (e.g. in the absence of another active ingredient). It is also to be understood that pharmaceutically acceptable salts of the active pharmaceutical ingredients may be included in the compositions provided herein.

In some embodiments, the active pharmaceutical ingredient is an immunosuppressant. An immunosuppressant as defined herein is an agent that can suppress or prevent the immune response Immunosuppressants are generally used when a normal immune response is undesirable (e.g. organ transplantation, autoimmune diseases). Examples of immunosuppressants suitable for the compositions and methods according to the embodiments of the present invention are TNF-α inhibitors including thalidomide and lenalidomide; IL-2 inhibitors including abetimus and gusperimus; macrolides including cyclosporine and tacrolimus; purine and pyrimidine synthesis inhibitors including azathioprine, mycophenolic acid, leflunomide and teriflunomide. In some further embodiment, the immunosuppressant is cyclosporine. In some embodiments, the cyclosporine is cyclosporine A. In other embodiments, the immunosuppressant is any appropriate pharmaceutical salt, prodrug and/or analog of cyclosporine. In some embodiments, the cyclosporine is present in an amount approximately equal to or less than about 4% w/w. In some embodiments, the cyclosporine is present from about 0.0001 to about 4, from about 0.0005 to about 4, from about 0.001 to about 4, from about 0.005 to about 4, from about 0.01 to about 4, from about 0.02 to about 4, from about 0.04 to about 4, from about 0.06 to about 4, from about 0.08 to about 4, from about 0.1 to about 4, from about 0.2 to about 4, from about 0.4 to about 4, from about 0.6 to about 4, from about 0.8 to about 4, from about 1 to about 4, from about 2 to about 4, from about 3 to about 4, from about 0.0001 to about 3, from about 0.0005 to about 3, from about 0.001 to about 3, from about 0.005 to about 3, from about 0.01 to about 3, from about 0.02 to about 3, from about 0.04 to about 3, from about 0.06 to about 3, from about 0.08 to about 3, from about 0.1 to about 3, from about 0.2 to about 3, from about 0.4 to about 3, from about 0.6 to about 3, from about 0.8 to about 3, from about 1 to about 3, from about 2 to about 3, from about 0.0001 to about 2, from about 0.0005 to about 2, from about 0.001 to about 2, from about 0.005 to about 2, from about 0.01 to about 2, from about 0.02 to about 2, from about 0.04 to about 2, from about 0.06 to about 2, from about 0.08 to about 2, from about 0.1 to about 2, from about 0.2 to about 2, from about 0.4 to about 2, from about 0.6 to about 2, from about 0.8 to about 2, from about 1 to about 2, from about 0.0001 to about 1, from about 0.0005 to about 1, from about 0.001 to about 1, from about 0.005 to about 1, from about 0.01 to about 1, from about 0.02 to about 1, from about 0.04 to about 1, from about 0.06 to about 1, from about 0.08 to about 1, from about 0.1 to about 1, from about 0.2 to about 1, from about 0.4 to about 1, from about 0.6 to about 1, or from about 0.8 to about 1% (w/w). The numerical values above represent amounts of the active ingredient in % (w/w).

In some embodiments, the cyclosporine is present from about 0.0001 to about 0.8, from about 0.0005 to about 0.8, from about 0.001 to about 0.8, from about 0.005 to about 0.8, from about 0.01 to about 0.8, from about 0.02 to about 0.8, from about 0.04 to about 0.8, from about 0.06 to about 0.8, from about 0.08 to about 0.8, from about 0.1 to about 0.8, from about 0.2 to about 0.8, from about 0.4 to about 0.8, from about 0.6 to about 0.8, from about 0.0001 to about 0.6, from about 0.0005 to about 0.6, from about 0.001 to about 0.6, from about 0.005 to about 0.6, from about 0.01 to about 0.6, from about 0.02 to about 0.6, from about 0.04 to about 0.6, from about 0.06 to about 0.6, from about 0.08 to about 0.6, from about 0.1 to about 0.6, from about 0.2 to about 0.6, from about 0.4 to about 0.6, from about 0.0001 to about 0.4, from about 0.0005 to about 0.4, from about 0.001 to about 0.4, from about 0.005 to about 0.4, from about 0.01 to about 0.4, from about 0.02 to about 0.4, from about 0.04 to about 0.4, from about 0.06 to about 0.4, from about 0.08 to about 0.4, from about 0.1 to about 0.4, from about 0.2 to about 0.4, from about 0.0001 to about 0.2, from about 0.0005 to about 0.2, from about 0.001 to about 0.2, from about 0.005 to about 0.2, from about 0.01 to about 0.2, from about 0.02 to about 0.2, from about 0.04 to about 0.2, from about 0.06 to about 0.2, from about 0.08 to about 0.2, or from about 0.1 to about 0.2% (w/w). The numerical values above represent amounts of the active ingredient in % (w/w).

In some embodiments, the cyclosporine is present from about 0.0001 to about 0.1, from about 0.0005 to about 0.1, from about 0.001 to about 0.1, from about 0.005 to about 0.1, from about 0.01 to about 0.1, from about 0.02 to about 0.1, from about 0.04 to about 0.1, from about 0.06 to about 0.1, from about 0.08 to about 0.1, from about 0.0001 to about 0.08, from about 0.0005 to about 0.08, from about 0.001 to about 0.08, from about 0.005 to about 0.08, from about 0.01 to about 0.08, from about 0.02 to about 0.08, from about 0.04 to about 0.08, from about 0.06 to about 0.08, from about 0.0001 to about 0.06, from about 0.0005 to about 0.06, from about 0.001 to about 0.06, from about 0.005 to about 0.06, from about 0.01 to about 0.06, from about 0.02 to about 0.06, from about 0.04 to about 0.06, from about 0.0001 to about 0.04, from about 0.0005 to about 0.04, from about 0.001 to about 0.04, from about 0.005 to about 0.04, from about 0.01 to about 0.04, from about 0.02 to about 0.04, from about 0.0001 to about 0.02, from about 0.0005 to about 0.02, from about 0.001 to about 0.02, from about 0.005 to about 0.02, from about 0.01 to about 0.02, from about 0.0001 to about 0.01, from about 0.0005 to about 0.01, from about 0.001 to about 0.01, from about 0.005 to about 0.01, from about 0.0001 to about 0.005, from about 0.0005 to about 0.005, from about 0.001 to about 0.005, from about 0.0001 to about 0.001, from about 0.0005 to about 0.001, or from about 0.0001 to about 0.0005% (w/w). In some embodiments, the cyclosporine is present at about 0.0001, 0.0005, 0.001, 0.005, 0.01, 0.02, 0.04, 0.06, 0.08, 0.1, 0.2, 0.4, 0.6, 0.8, 1, 2, 3, or 4% (w/w). In some embodiments, the cyclosporine is present in an amount of about 0.001% w/w. The numerical values above represent amounts of the active ingredient in % (w/w).

In some embodiments, the immunosuppressant is tacrolimus. In other embodiments, the immunosuppressant is any appropriate pharmaceutical salt, prodrug and/or analog of tacrolimus. In some embodiments, the tacrolimus is present in an amount approximately equal to or less than about 0.1% w/w. In some embodiments, the tacrolimus is present from about 0.01 to about 0.1, from about 0.02 to about 0.1, from about 0.03 to about 0.1, from about 0.04 to about 0.1, from about 0.05 to about 0.1, from about 0.06 to about 0.1, from about 0.07 to about 0.1, from about 0.08 to about 0.1, from about 0.09 to about 0.1, from about 0.02 to about 0.09, from about 0.03 to about 0.09, from about 0.04 to about 0.09, from about 0.05 to about 0.09, from about 0.06 to about 0.09, from about 0.07 to about 0.09, from about 0.08 to about 0.09, from about 0.02 to about 0.08, from about 0.03 to about 0.08, from about 0.04 to about 0.08, from about 0.05 to about 0.08, from about 0.06 to about 0.08, from about 0.07 to about 0.08, from about 0.02 to about 0.07, from about 0.03 to about 0.07, from about 0.04 to about 0.07, from about 0.05 to about 0.07, from about 0.06 to about 0.07, from about 0.02 to about 0.06, from about 0.03 to about 0.06, from about 0.04 to about 0.06, from about 0.05 to about 0.06, from about 0.02 to about 0.05, from about 0.03 to about 0.05, from about 0.04 to about 0.05, from about 0.02 to about 0.04, from about 0.03 to about 0.04, or from about 0.02 to about 0.03% (w/w). In some embodiments, the tacrolimus is present at about 0.01, 0.02, 0.03, 0.04, 0.05, 0.06, 0.07, 0.08, 0.09, or 0.1% (w/w). In some embodiments, the tacrolimus is present in an amount of about 0.01% w/w. The numerical values above represent amounts of the active ingredient in % (w/w).

In some embodiments, the active pharmaceutical ingredient is a vasodilator agent. A vasodilator agent as defined herein is an agent that widens the blood vessels, which in turn decreases resistance to blood flow and lowers blood pressure. Based on their mechanism of action vasodilators (i.e. vasodilator agents) can be calcium channel blockers or alpha adrenergic antagonists. Examples of calcium channel blockers are amlodipine, felodipine, isradipine, lecranidipine, nicardipine, nifedipine, nimodipine, diltiazem and verapamil. Examples of adrenergic antagonists are doxazosin, phentolamine, phenoxybenzamine, terazosin, tolazoline, and idazoxan. In some embodiments, the vasodilator agent is an alpha adrenergic antagonist. In some further embodiments, the alpha adrenergic antagonist is phentolamine. In other embodiments, the alpha adrenergic antagonist is any appropriate pharmaceutical salt, prodrug and/or analog of phentolamine. In some embodiments, the phentolamine is present in an amount approximately equal to or less than about 4% w/w. In some embodiments, the phentolamine is present from about 0.0001 to about 4, from about 0.0005 to about 4, from about 0.001 to about 4, from about 0.005 to about 4, from about 0.01 to about 4, from about 0.02 to about 4, from about 0.04 to about 4, from about 0.06 to about 4, from about 0.08 to about 4, from about 0.1 to about 4, from about 0.2 to about 4, from about 0.4 to about 4, from about 0.6 to about 4, from about 0.8 to about 4, from about 1 to about 4, from about 2 to about 4, from about 3 to about 4, from about 0.0001 to about 3, from about 0.0005 to about 3, from about 0.001 to about 3, from about 0.005 to about 3, from about 0.01 to about 3, from about 0.02 to about 3, from about 0.04 to about 3, from about 0.06 to about 3, from about 0.08 to about 3, from about 0.1 to about 3, from about 0.2 to about 3, from about 0.4 to about 3, from about 0.6 to about 3, from about 0.8 to about 3, from about 1 to about 3, from about 2 to about 3, from about 0.0001 to about 2, from about 0.0005 to about 2, from about 0.001 to about 2, from about 0.005 to about 2, from about 0.01 to about 2, from about 0.02 to about 2, from about 0.04 to about 2, from about 0.06 to about 2, from about 0.08 to about 2, from about 0.1 to about 2, from about 0.2 to about 2, from about 0.4 to about 2, from about 0.6 to about 2, from about 0.8 to about 2, from about 1 to about 2, from about 0.0001 to about 1, from about 0.0005 to about 1, from about 0.001 to about 1, from about 0.005 to about 1, from about 0.01 to about 1, from about 0.02 to about 1, from about 0.04 to about 1, from about 0.06 to about 1, from about 0.08 to about 1, from about 0.1 to about 1, from about 0.2 to about 1, from about 0.4 to about 1, from about 0.6 to about 1, or from about 0.8 to about 1% (w/w). The numerical values above represent amounts of the active ingredient in % (w/w).

In some embodiments, the phentolamine is present from about 0.0001 to about 0.8, from about 0.0005 to about 0.8, from about 0.001 to about 0.8, from about 0.005 to about 0.8, from about 0.01 to about 0.8, from about 0.02 to about 0.8, from about 0.04 to about 0.8, from about 0.06 to about 0.8, from about 0.08 to about 0.8, from about 0.1 to about 0.8, from about 0.2 to about 0.8, from about 0.4 to about 0.8, from about 0.6 to about 0.8, from about 0.0001 to about 0.6, from about 0.0005 to about 0.6, from about 0.001 to about 0.6, from about 0.005 to about 0.6, from about 0.01 to about 0.6, from about 0.02 to about 0.6, from about 0.04 to about 0.6, from about 0.06 to about 0.6, from about 0.08 to about 0.6, from about 0.1 to about 0.6, from about 0.2 to about 0.6, from about 0.4 to about 0.6, from about 0.0001 to about 0.4, from about 0.0005 to about 0.4, from about 0.001 to about 0.4, from about 0.005 to about 0.4, from about 0.01 to about 0.4, from about 0.02 to about 0.4, from about 0.04 to about 0.4, from about 0.06 to about 0.4, from about 0.08 to about 0.4, from about 0.1 to about 0.4, from about 0.2 to about 0.4, from about 0.0001 to about 0.2, from about 0.0005 to about 0.2, from about 0.001 to about 0.2, from about 0.005 to about 0.2, from about 0.01 to about 0.2, from about 0.02 to about 0.2, from about 0.04 to about 0.2, from about 0.06 to about 0.2, from about 0.08 to about 0.2, or from about 0.1 to about 0.2% (w/w). The numerical values above represent amounts of the active ingredient in % (w/w).

In some embodiments, the phentolamine is present from about 0.0001 to about 0.1, from about 0.0005 to about 0.1, from about 0.001 to about 0.1, from about 0.005 to about 0.1, from about 0.01 to about 0.1, from about 0.02 to about 0.1, from about 0.04 to about 0.1, from about 0.06 to about 0.1, from about 0.08 to about 0.1, from about 0.0001 to about 0.08, from about 0.0005 to about 0.08, from about 0.001 to about 0.08, from about 0.005 to about 0.08, from about 0.01 to about 0.08, from about 0.02 to about 0.08, from about 0.04 to about 0.08, from about 0.06 to about 0.08, from about 0.0001 to about 0.06, from about 0.0005 to about 0.06, from about 0.001 to about 0.06, from about 0.005 to about 0.06, from about 0.01 to about 0.06, from about 0.02 to about 0.06, from about 0.04 to about 0.06, from about 0.0001 to about 0.04, from about 0.0005 to about 0.04, from about 0.001 to about 0.04, from about 0.005 to about 0.04, from about 0.01 to about 0.04, from about 0.02 to about 0.04, from about 0.0001 to about 0.02, from about 0.0005 to about 0.02, from about 0.001 to about 0.02, from about 0.005 to about 0.02, from about 0.01 to about 0.02, from about 0.0001 to about 0.01, from about 0.0005 to about 0.01, from about 0.001 to about 0.01, from about 0.005 to about 0.01, from about 0.0001 to about 0.005, from about 0.0005 to about 0.005, from about 0.001 to about 0.005, from about 0.0001 to about 0.001, from about 0.0005 to about 0.001, or from about 0.0001 to about 0.0005% (w/w). In some embodiments, the phentolamine is present at about 0.0001, 0.0005, 0.001, 0.005, 0.01, 0.02, 0.04, 0.06, 0.08, 0.1, 0.2, 0.4, 0.6, 0.8, 1, 2, 3, or 4% (w/w). In some embodiments, the phentolamine is present in an amount of about 0.001% w/w. The numerical values above represent amounts of the active ingredient in % (w/w).

In some embodiments, the active pharmaceutical ingredient is an anti-inflammatory agent. Anti-inflammatory agents as defined herein are agents capable of reducing inflammation. Anti-inflammatory agents include steroids (e.g. glucocorticoids, androgens), non-steroidal anti-inflammatory agent (e.g. non-steroidal anti-inflammatory drugs (NSAID)) and immune selective anti-inflammatory derivatives (ImSAIDs). In some embodiments, the anti-inflammatory agent is a non-steroidal anti-inflammatory agent. Non-steroidal anti-inflammatory agents include drugs with analgesic and fever-reducing effects, which inhibit the synthesis of prostaglandins. Examples of non-steroidal anti-inflammatory agents include aspirin, ibuprofen, naproxen, etodolac, ketorolac, tenoxicam, lornoxicam, celecoxib, and nemesolide. In some embodiments, the non-steroidal anti-inflammatory agent is ketorolac. In other embodiments, the non-steroidal anti-inflammatory agent is any appropriate pharmaceutical salt, prodrug and/or analog of ketorolac. In some embodiments, the ketorolac is present in an amount approximately equal to or less than about 2% w/w. In some embodiments, the ketorolac is present from about 0.001 to about 2, from about 0.004 to about 2, from about 0.008 to about 2, from about 0.01 to about 2, from about 0.04 to about 2, from about 0.08 to about 2, from about 0.1 to about 2, from about 0.4 to about 2, from about 0.8 to about 2, from about 1 to about 2, from about 1.4 to about 2, from about 1.8 to about 2, from about 0.001 to about 1.8, from about 0.004 to about 1.8, from about 0.008 to about 1.8, from about 0.01 to about 1.8, from about 0.04 to about 1.8, from about 0.08 to about 1.8, from about 0.1 to about 1.8, from about 0.4 to about 1.8, from about 0.8 to about 1.8, from about 1 to about 1.8, or from about 1.4 to about 1.8% w/w. The numerical values above represent amounts of the active ingredient in % (w/w).

In some embodiments, the ketorolac is present from about 0.001 to about 1.4, from about 0.004 to about 1.4, from about 0.008 to about 1.4, from about 0.01 to about 1.4, from about 0.04 to about 1.4, from about 0.08 to about 1.4, from about 0.1 to about 1.4, from about 0.4 to about 1.4, from about 0.8 to about 1.4, from about 1 to about 1.4, from about 0.001 to about 1, from about 0.004 to about 1, from about 0.008 to about 1, from about 0.01 to about 1, from about 0.04 to about 1, from about 0.08 to about 1, from about 0.1 to about 1, from about 0.4 to about 1, from about 0.8 to about 1, from about 0.001 to about 0.8, from about 0.004 to about 0.8, from about 0.008 to about 0.8, from about 0.01 to about 0.8, from about 0.04 to about 0.8, from about 0.08 to about 0.8, from about 0.1 to about 0.8, from about 0.4 to about 0.8, from about 0.001 to about 0.4, from about 0.004 to about 0.4, from about 0.008 to about 0.4, from about 0.01 to about 0.4, from about 0.04 to about 0.4, from about 0.08 to about 0.4, from about 0.1 to about 0.4, from about 0.001 to about 0.1, from about 0.004 to about 0.1, from about 0.008 to about 0.1, from about 0.01 to about 0.1, from about 0.04 to about 0.1, from about 0.08 to about 0.1, from about 0.001 to about 0.08, from about 0.004 to about 0.08, from about 0.008 to about 0.08, from about 0.01 to about 0.08, from about 0.04 to about 0.08, from about 0.001 to about 0.04, from about 0.004 to about 0.04, from about 0.008 to about 0.04, from about 0.01 to about 0.04, from about 0.001 to about 0.01, from about 0.004 to about 0.01, from about 0.008 to about 0.01, from about 0.001 to about 0.008, from about 0.004 to about 0.008, or from about 0.001 to about 0.0045 w/w. In some embodiments, the ketorolac is present at about 0.001, 0.004, 0.008, 0.01, 0.04, 0.08, 0.1, 0.4, 0.8, 1, 1.4, 1.8 or 2% (w/w). In some embodiments, the ketorolac is present in an amount of about 0.01% w/w. The numerical values above represent amounts of the active ingredient in % (w/w).

In some embodiments, the anti-inflammatory agent is an androgen. Androgens are steroid hormones that stimulate or control the development and maintenance of male characteristics in vertebrates by binding to androgen receptors. Androgens are produced naturally by the testis and are required for the activity of the accessory male sex organs and the development of male secondary sex characteristics. Examples of androgens include testosterone, dihydrotestosterone, dehydroepiandrosterone, androsterone and androstenedione. In some further embodiments, the anti-inflammatory agent is testosterone. In other embodiments, the anti-inflammatory agent is any appropriate pharmaceutical salt, prodrug and/or analog of testosterone. In some embodiments, the testosterone is present in an amount approximately equal to or less than about 5% w/w. In some embodiments, the testosterone is present from about 0.001 to about 5, from about 0.005 to about 5, from about 0.01 to about 5, from about 0.05 to about 5, from about 0.1 to about 5, from about 0.5 to about 5, from about 1 to about 5, from about 1.5 to about 5, from about 2 to about 5, from about 2.5 to about 5, from about 3 to about 5, from about 3.5 to about 5, from about 4 to about 5, from about 4.5, from about 0.001 to about 4.5, from about 0.005 to about 4.5, from about 0.01 to about 4.5, from about 0.05 to about 4.5, from about 0.1 to about 4.5, from about 0.5 to about 4.5, from about 1 to about 4.5, from about 1.5 to about 4.5, from about 2 to about 4.5, from about 2.5 to about 4.5, from about 3 to about 4.5, from about 3.5 to about 4.5, from about 4 to about 4.5, from about 0.001 to about 4, from about 0.005 to about 4, or from about 0.01 to about 4% w/w. The numerical values above represent amounts of the active ingredient in % (w/w).

In some embodiments, the testosterone is present from about 0.05 to about 4, from about 0.1 to about 4, from about 0.5 to about 4, from about 1 to about 4, from about 1.5 to about 4, from about 2 to about 4, from about 2.5 to about 4, from about 3 to about 4, from about 3.5 to about 4, from about 0.001 to about 3.5, from about 0.005 to about 3.5, from about 0.01 to about 3.5, from about 0.05 to about 3.5, from about 0.1 to about 3.5, from about 0.5 to about 3.5, from about 1 to about 3.5, from about 1.5 to about 3.5, from about 2 to about 3.5, from about 2.5 to about 3.5, from about 3 to about 3.5, from about 0.001 to about 3, from about 0.005 to about 3, from about 0.01 to about 3, from about 0.05 to about 3, from about 0.1 to about 3, from about 0.5 to about 3, from about 1 to about 3, from about 1.5 to about 3, from about 2 to about 3, from about 2.5 to about 3, from about 0.001 to about 2.5, from about 0.005 to about 2.5, from about 0.01 to about 2.5, from about 0.05 to about 2.5, from about 0.1 to about 2.5, from about 0.5 to about 2.5, from about 1 to about 2.5, from about 1.5 to about 2.5, from about 2 to about 2.5, from about 0.001 to about 2, from about 0.005 to about 2, from about 0.01 to about 2, from about 0.05 to about 2, from about 0.1 to about 2, from about 0.5 to about 2, from about 1 to about 2, from about 1.5 to about 2, from about 0.001 to about 1.5, from about 0.005 to about 1.5, from about 0.01 to about 1.5, from about 0.05 to about 1.5, from about 0.1 to about 1.5, from about 0.5 to about 1.5, from about 1 to about 1.5, from about 0.001 to about 1, from about 0.005 to about 1, from about 0.01 to about 1, from about 0.05 to about 1, from about 0.1 to about 1, from about 0.5 to about 1, from about 0.001 to about 0.5, from about 0.005 to about 0.5, from about 0.01 to about 0.5, from about 0.05 to about 0.5, from about 0.1 to about 0.5, from about 0.001 to about 0.1, from about 0.005 to about 0.1, from about 0.01 to about 0.1, from about 0.05 to about 0.1, from about 0.001 to about 0.05, from about 0.005 to about 0.05, from about 0.01 to about 0.05, or from about 0.001 to about 0.005% (w/w). In some embodiments, the testosterone is present at about 0.001, 0.005, 0.01, 0.05, 0.1, 0.5, 1, 1.5, 2, 2.5, 3, 3.5, 4, 4.5, or 5% (w/w). In some embodiments, the testosterone is present in an amount of about 0.001% w/w. The numerical values above represent amounts of the active ingredient in % (w/w).

In some embodiments, the anti-inflammatory agent is dihydrotestosterone. In other embodiments, the anti-inflammatory agent is any appropriate pharmaceutical salt, prodrug and/or analog of dihydrotestosterone. In some embodiments, the dihydrotestosterone is present in an amount approximately equal to or less than about 5% w/w. In some embodiments, the dihydrotestosterone is present from about 0.001 to about 5, from about 0.005 to about 5, from about 0.01 to about 5, from about 0.05 to about 5, from about 0.1 to about 5, from about 0.5 to about 5, from about 1 to about 5, from about 1.5 to about 5, from about 2 to about 5, from about 2.5 to about 5, from about 3 to about 5, from about 3.5 to about 5, from about 4 to about 5, from about 4.5, from about 0.001 to about 4.5, from about 0.005 to about 4.5, from about 0.01 to about 4.5, from about 0.05 to about 4.5, from about 0.1 to about 4.5, from about 0.5 to about 4.5, from about 1 to about 4.5, from about 1.5 to about 4.5, from about 2 to about 4.5, from about 2.5 to about 4.5, from about 3 to about 4.5, from about 3.5 to about 4.5, or from about 4 to about 4.5% w/w. The numerical values above represent amounts of the active ingredient in % (w/w).

In some embodiments, the dihydrotestosterone is present from about 0.001 to about 4, from about 0.005 to about 4, from about 0.01 to about 4, from about 0.05 to about 4, from about 0.1 to about 4, from about 0.5 to about 4, from about 1 to about 4, from about 1.5 to about 4, from about 2 to about 4, from about 2.5 to about 4, from about 3 to about 4, from about 3.5 to about 4, from about 0.001 to about 3.5, from about 0.005 to about 3.5, from about 0.01 to about 3.5, from about 0.05 to about 3.5, from about 0.1 to about 3.5, from about 0.5 to about 3.5, from about 1 to about 3.5, from about 1.5 to about 3.5, from about 2 to about 3.5, from about 2.5 to about 3.5, from about 3 to about 3.5, from about 0.001 to about 3, from about 0.005 to about 3, from about 0.01 to about 3, from about 0.05 to about 3, from about 0.1 to about 3, from about 0.5 to about 3, from about 1 to about 3, from about 1.5 to about 3, from about 2 to about 3, from about 2.5 to about 3, from about 0.001 to about 2.5, from about 0.005 to about 2.5, from about 0.01 to about 2.5, from about 0.05 to about 2.5, from about 0.1 to about 2.5, from about 0.5 to about 2.5, from about 1 to about 2.5, from about 1.5 to about 2.5, from about 2 to about 2.5, from about 0.001 to about 2, from about 0.005 to about 2, from about 0.01 to about 2, from about 0.05 to about 2, from about 0.1 to about 2, from about 0.5 to about 2, from about 1 to about 2, from about 1.5 to about 2, from about 0.001 to about 1.5, from about 0.005 to about 1.5, from about 0.01 to about 1.5, from about 0.05 to about 1.5, from about 0.1 to about 1.5, from about 0.5 to about 1.5, from about 1 to about 1.5, from about 0.001 to about 1, from about 0.005 to about 1, from about 0.01 to about 1, from about 0.05 to about 1, from about 0.1 to about 1, from about 0.5 to about 1, from about 0.001 to about 0.5, from about 0.005 to about 0.5, from about 0.01 to about 0.5, from about 0.05 to about 0.5, from about 0.1 to about 0.5, from about 0.001 to about 0.1, from about 0.005 to about 0.1, from about 0.01 to about 0.1, from about 0.05 to about 0.1, from about 0.001 to about 0.05, from about 0.005 to about 0.05, from about 0.01 to about 0.05, or from about 0.001 to about 0.005% (w/w). In some embodiments, the dihydrotestosterone is present at about 0.001, 0.005, 0.01, 0.05, 0.1, 0.5, 1, 1.5, 2, 2.5, 3, 3.5, 4, 4.5, or 5% (w/w). In some embodiments, the dihydrotestosterone is present in an amount of about 0.001% w/w. The numerical values above represent amounts of the active ingredient in % (w/w).

In some embodiments, the anti-inflammatory agent is testosterone propionate. In other embodiments, the anti-inflammatory agent is any appropriate pharmaceutical salt, prodrug and/or analog of testosterone propionate. In some embodiments, the testosterone propionate is present in an amount approximately equal to or less than about 5% w/w. In some embodiments, the testosterone propionate is present from about 0.001 to about 5, from about 0.005 to about 5, from about 0.01 to about 5, from about 0.05 to about 5, from about 0.1 to about 5, from about 0.5 to about 5, from about 1 to about 5, from about 1.5 to about 5, from about 2 to about 5, from about 2.5 to about 5, from about 3 to about 5, from about 3.5 to about 5, from about 4 to about 5, from about 4.5, from about 0.001 to about 4.5, from about 0.005 to about 4.5, from about 0.01 to about 4.5, from about 0.05 to about 4.5, from about 0.1 to about 4.5, from about 0.5 to about 4.5, from about 1 to about 4.5, from about 1.5 to about 4.5, from about 2 to about 4.5, from about 2.5 to about 4.5, from about 3 to about 4.5, from about 3.5 to about 4.5, from about 4 to about 4.5, from about 0.001 to about 4, from about 0.005 to about 4, from about 0.01 to about 4, from about 0.05 to about 4, from about 0.1 to about 4, from about 0.5 to about 4, from about 1 to about 4, from about 1.5 to about 4, from about 2 to about 4, from about 2.5 to about 4, from about 3 to about 4, from about 3.5 to about 4% w/w. The numerical values above represent amounts of the active ingredient in % (w/w).

In some embodiments, the testosterone propionate is present from about 0.001 to about 3.5, from about 0.005 to about 3.5, from about 0.01 to about 3.5, from about 0.05 to about 3.5, from about 0.1 to about 3.5, from about 0.5 to about 3.5, from about 1 to about 3.5, from about 1.5 to about 3.5, from about 2 to about 3.5, from about 2.5 to about 3.5, from about 3 to about 3.5, from about 0.001 to about 3, from about 0.005 to about 3, from about 0.01 to about 3, from about 0.05 to about 3, from about 0.1 to about 3, from about 0.5 to about 3, from about 1 to about 3, from about 1.5 to about 3, from about 2 to about 3, from about 2.5 to about 3, from about 0.001 to about 2.5, from about 0.005 to about 2.5, from about 0.01 to about 2.5, from about 0.05 to about 2.5, from about 0.1 to about 2.5, from about 0.5 to about 2.5, from about 1 to about 2.5, from about 1.5 to about 2.5, from about 2 to about 2.5, from about 0.001 to about 2, from about 0.005 to about 2, from about 0.01 to about 2, from about 0.05 to about 2, from about 0.1 to about 2, from about 0.5 to about 2, from about 1 to about 2, from about 1.5 to about 2, from about 0.001 to about 1.5, from about 0.005 to about 1.5, from about 0.01 to about 1.5, from about 0.05 to about 1.5, from about 0.1 to about 1.5, from about 0.5 to about 1.5, from about 1 to about 1.5, from about 0.001 to about 1, from about 0.005 to about 1, from about 0.01 to about 1, from about 0.05 to about 1, from about 0.1 to about 1, from about 0.5 to about 1, from about 0.001 to about 0.5, from about 0.005 to about 0.5, from about 0.01 to about 0.5, from about 0.05 to about 0.5, from about 0.1 to about 0.5, from about 0.001 to about 0.1, from about 0.005 to about 0.1, from about 0.01 to about 0.1, from about 0.05 to about 0.1, from about 0.001 to about 0.05, from about 0.005 to about 0.05, from about 0.01 to about 0.05, or from about 0.001 to about 0.005% (w/w). In some embodiments, the testosterone propionate is present at about 0.001, 0.005, 0.01, 0.05, 0.1, 0.5, 1, 1.5, 2, 2.5, 3, 3.5, 4, 4.5, or 5% (w/w). In some embodiments, the testosterone propionate is present in an amount of about 0.001% w/w. The numerical values above represent amounts of the active ingredient in % (w/w).

The anti-inflammatory agent provided herein may be dexamethasone or prednisolone. In some embodiments, the anti-inflammatory agent is dexamethasone. In other embodiments, the anti-inflammatory agent is any appropriate pharmaceutical salt, prodrug and/or analog of dexamethasone. In some embodiments, the dexamethasone is present in an amount approximately equal to or less than about 5% w/w. In some embodiments, the dexamethasone is present from about 0.001 to about 5, from about 0.005 to about 5, from about 0.01 to about 5, from about 0.05 to about 5, from about 0.1 to about 5, from about 0.5 to about 5, from about 1 to about 5, from about 1.5 to about 5, from about 2 to about 5, from about 2.5 to about 5, from about 3 to about 5, from about 3.5 to about 5, from about 4 to about 5, from about 4.5, from about 0.001 to about 4.5, from about 0.005 to about 4.5, from about 0.01 to about 4.5, from about 0.05 to about 4.5, from about 0.1 to about 4.5, from about 0.5 to about 4.5, from about 1 to about 4.5, from about 1.5 to about 4.5, from about 2 to about 4.5, from about 2.5 to about 4.5, from about 3 to about 4.5, from about 3.5 to about 4.5, from about 4 to about 4.5, from about 0.001 to about 4, from about 0.005 to about 4, from about 0.01 to about 4, from about 0.05 to about 4, from about 0.1 to about 4, from about 0.5 to about 4, from about 1 to about 4, from about 1.5 to about 4, from about 2 to about 4, from about 2.5 to about 4, from about 3 to about 4, from about 3.5 to about 4, from about 0.001 to about 3.5, from about 0.005 to about 3.5, from about 0.01 to about 3.5, from about 0.05 to about 3.5, from about 0.1 to about 3.5, from about 0.5 to about 3.5, from about 1 to about 3.5, from about 1.5 to about 3.5, from about 2 to about 3.5, from about 2.5 to about 3.5, from about 3 to about 3.5, from about 0.001 to about 3, from about 0.005 to about 3, from about 0.01 to about 3, from about 0.05 to about 3, from about 0.1 to about 3, from about 0.5 to about 3, from about 1 to about 3, from about 1.5 to about 3, from about 2 to about 3, from about 2.5 to about 3, from about 0.001 to about 2.5, from about 0.005 to about 2.5, from about 0.01 to about 2.5, from about 0.05 to about 2.5, from about 0.1 to about 2.5, from about 0.5 to about 2.5, from about 1 to about 2.5, from about 1.5 to about 2.5, from about 2 to about 2.5, from about 0.001 to about 2, from about 0.005 to about 2, from about 0.01 to about 2, from about 0.05 to about 2, from about 0.1 to about 2, from about 0.5 to about 2, from about 1 to about 2, from about 1.5 to about 2, from about 0.001 to about 1.5, from about 0.005 to about 1.5, from about 0.01 to about 1.5, from about 0.05 to about 1.5, from about 0.1 to about 1.5, from about 0.5 to about 1.5, from about 1 to about 1.5, from about 0.001 to about 1, from about 0.005 to about 1, from about 0.01 to about 1, from about 0.05 to about 1, from about 0.1 to about 1, from about 0.5 to about 1, from about 0.001 to about 0.5, from about 0.005 to about 0.5, from about 0.01 to about 0.5, from about 0.05 to about 0.5, from about 0.1 to about 0.5, from about 0.001 to about 0.1, from about 0.005 to about 0.1, from about 0.01 to about 0.1, from about 0.05 to about 0.1, from about 0.001 to about 0.05, from about 0.005 to about 0.05, from about 0.01 to about 0.05, or from about 0.001 to about 0.005% (w/w). In some embodiments, the dexamethasone is present at about 0.001, 0.005, 0.01, 0.05, 0.1, 0.5, 1, 1.5, 2, 2.5, 3, 3.5, 4, 4.5, or 5% (w/w). In some embodiments, the dexamethasone is present in an amount of about 0.001% w/w. The numerical values above represent amounts of the active ingredient in % (w/w).

In other embodiments, the anti-inflammatory agent is prednisolone. In other embodiments, the anti-inflammatory agent is any appropriate pharmaceutical salt, prodrug and/or analog of prednisolone. In some embodiments, the prednisolone is present in an amount approximately equal to or less than about 5% w/w. In some embodiments, the prednisolone is present from about 0.001 to about 5, from about 0.005 to about 5, from about 0.01 to about 5, from about 0.05 to about 5, from about 0.1 to about 5, from about 0.5 to about 5, from about 1 to about 5, from about 1.5 to about 5, from about 2 to about 5, from about 2.5 to about 5, from about 3 to about 5, from about 3.5 to about 5, from about 4 to about 5, from about 4.5, from about 0.001 to about 4.5, from about 0.005 to about 4.5, from about 0.01 to about 4.5, from about 0.05 to about 4.5, from about 0.1 to about 4.5, from about 0.5 to about 4.5, from about 1 to about 4.5, from about 1.5 to about 4.5, from about 2 to about 4.5, from about 2.5 to about 4.5, from about 3 to about 4.5, from about 3.5 to about 4.5, from about 4 to about 4.5, from about 0.001 to about 4, from about 0.005 to about 4, from about 0.01 to about 4, from about 0.05 to about 4, from about 0.1 to about 4, from about 0.5 to about 4, from about 1 to about 4, from about 1.5 to about 4, from about 2 to about 4, from about 2.5 to about 4, from about 3 to about 4, from about 3.5 to about 4, from about 0.001 to about 3.5, from about 0.005 to about 3.5, from about 0.01 to about 3.5, from about 0.05 to about 3.5, from about 0.1 to about 3.5, from about 0.5 to about 3.5, from about 1 to about 3.5, from about 1.5 to about 3.5, from about 2 to about 3.5, from about 2.5 to about 3.5, from about 3 to about 3.5, from about 0.001 to about 3, from about 0.005 to about 3, from about 0.01 to about 3, from about 0.05 to about 3, from about 0.1 to about 3, from about 0.5 to about 3, from about 1 to about 3, from about 1.5 to about 3, from about 2 to about 3, from about 2.5 to about 3, from about 0.001 to about 2.5, from about 0.005 to about 2.5, from about 0.01 to about 2.5, from about 0.05 to about 2.5, from about 0.1 to about 2.5, from about 0.5 to about 2.5, from about 1 to about 2.5, from about 1.5 to about 2.5, from about 2 to about 2.5, from about 0.001 to about 2, from about 0.005 to about 2, from about 0.01 to about 2, from about 0.05 to about 2, from about 0.1 to about 2, from about 0.5 to about 2, from about 1 to about 2, from about 1.5 to about 2, from about 0.001 to about 1.5, from about 0.005 to about 1.5, from about 0.01 to about 1.5, from about 0.05 to about 1.5, from about 0.1 to about 1.5, from about 0.5 to about 1.5, from about 1 to about 1.5, from about 0.001 to about 1, from about 0.005 to about 1, from about 0.01 to about 1, from about 0.05 to about 1, from about 0.1 to about 1, from about 0.5 to about 1, from about 0.001 to about 0.5, from about 0.005 to about 0.5, from about 0.01 to about 0.5, from about 0.05 to about 0.5, from about 0.1 to about 0.5, from about 0.001 to about 0.1, from about 0.005 to about 0.1, from about 0.01 to about 0.1, from about 0.05 to about 0.1, from about 0.001 to about 0.05, from about 0.005 to about 0.05, from about 0.01 to about 0.05, or from about 0.001 to about 0.005% (w/w). In some embodiments, the prednisolone is present at about 0.001, 0.005, 0.01, 0.05, 0.1, 0.5, 1, 1.5, 2, 2.5, 3, 3.5, 4, 4.5, or 5% (w/w). In some embodiments, the prednisolone is present in an amount of about 0.001% w/w. The numerical values above represent amounts of the active ingredient in % (w/w).

In some embodiments, the non-aqueous composition provided herein includes an EP2 receptor agonist. An EP2 receptor agonist is an agent capable of binding a prostaglandin E₂ receptor. EP2 receptor agonists typically increase an activity of a prostaglandin E₂ receptor. A prostaglandin E₂ receptor as used herein according to the ordinary usage in the art, and generally refers to a G-protein coupled receptor that may be bound by prostaglandin E₂. Prostaglandin E₂ is used according to its ordinary meaning and generally refers to a lipid mediator that is derived enzymatically from fatty acids. E₂ prostaglandins may have a variety of strong physiological effects, such as regulating the contraction and relaxation of smooth muscle tissue. Agents capable of binding a prostaglandin E₂ receptor are referred to herein as EP2 receptor agonists. Non limiting examples of EP2 receptor agonists are small molecules and chemical compounds. The non-aqueous compositions provided herein may include one or more EP2 receptor agonists. In some embodiments, the active pharmaceutical ingredient is an EP2 receptor agonist. In some embodiments, the EP2 receptor agonist is a compound of Formula

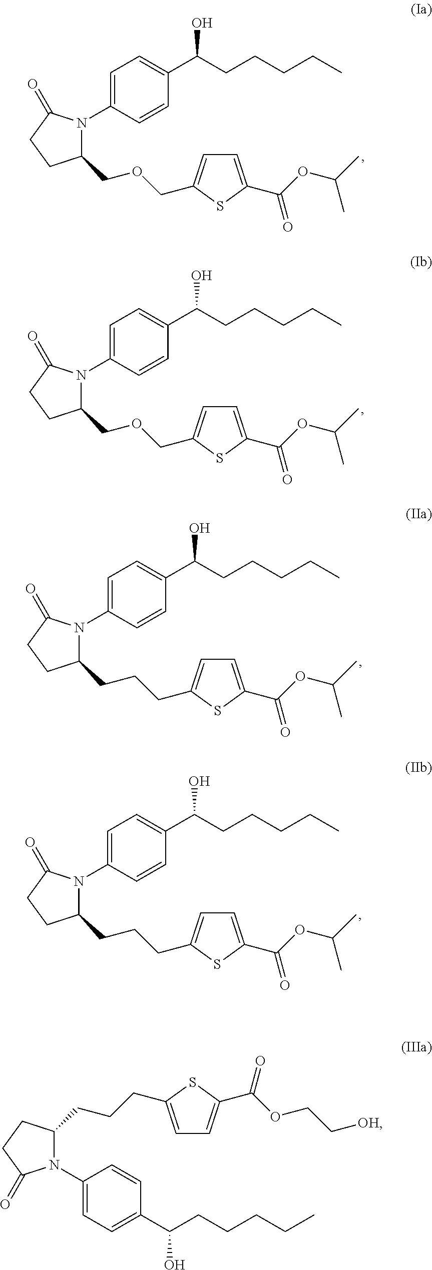

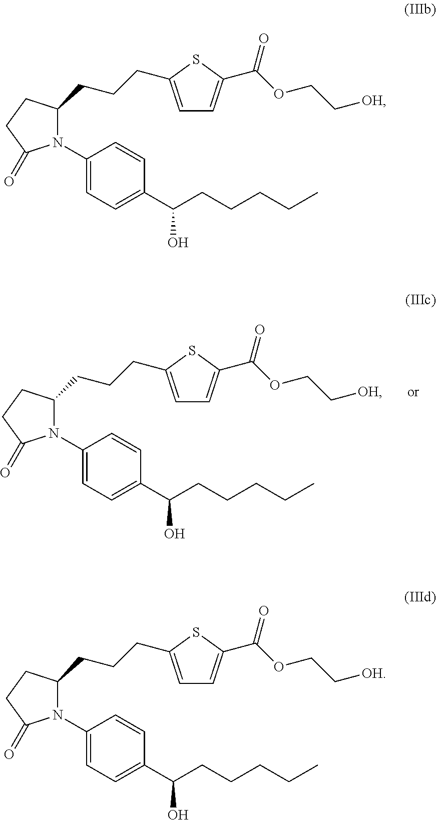

In some embodiments, the EP2 receptor agonist is a compound of Formula

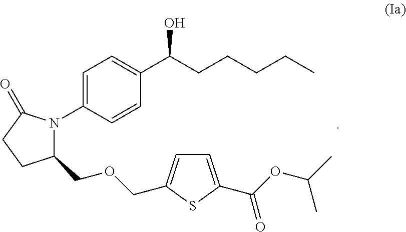

In other embodiments, the EP2 receptors agonist is any appropriate pharmaceutical salt, prodrug and/or analog of the compound of Formula (Ia). In some further embodiments, the EP2 receptor agonist is present in an amount approximately equal to or less than about 0.1% w/w. In some further embodiments, the EP2 receptor agonist is present from about 0.001 to about 0.1, from about 0.002 to about 0.1, from about 0.003 to about 0.1, from about 0.004 to about 0.1, from about 0.005 to about 0.1, from about 0.006 to about 0.1, from about 0.007 to about 0.1, from about 0.008 to about 0.1, from about 0.009 to about 0.1, from about 0.01 to about 0.1, from about 0.02 to about 0.1, from about 0.03 to about 0.1, from about 0.04 to about 0.1, from about 0.05 to about 0.1, from about 0.06 to about 0.1, from about 0.07 to about 0.1, from about 0.08 to about 0.1, from about 0.09 to about 0.1, from about 0.001 to about 0.08, from about 0.002 to about 0.08, from about 0.003 to about 0.08, from about 0.004 to about 0.08, from about 0.005 to about 0.08, from about 0.006 to about 0.08, from about 0.007 to about 0.08, from about 0.008 to about 0.08, from about 0.009 to about 0.08, from about 0.01 to about 0.08, from about 0.02 to about 0.08, from about 0.03 to about 0.08, from about 0.04 to about 0.08, from about 0.05 to about 0.08, from about 0.06 to about 0.08, from about 0.07 to about 0.08, from about 0.001 to about 0.06, from about 0.002 to about 0.06, from about 0.003 to about 0.06, from about 0.004 to about 0.06, from about 0.005 to about 0.06, from about 0.006 to about 0.06, from about 0.007 to about 0.06, from about 0.008 to about 0.06, from about 0.009 to about 0.06, from about 0.01 to about 0.06, from about 0.02 to about 0.06, from about 0.03 to about 0.06, from about 0.04 to about 0.06, from about 0.05 to about 0.06, from about 0.001 to about 0.04, from about 0.002 to about 0.04, from about 0.003 to about 0.04, from about 0.004 to about 0.04, from about 0.005 to about 0.04, from about 0.006 to about 0.04, from about 0.007 to about 0.04, from about 0.008 to about 0.04, from about 0.009 to about 0.04, from about 0.01 to about 0.04, from about 0.02 to about 0.04, from about 0.03 to about 0.04, from about 0.001 to about 0.02, from about 0.002 to about 0.02, from about 0.003 to about 0.02, from about 0.004 to about 0.02, from about 0.005 to about 0.02, from about 0.006 to about 0.02, from about 0.007 to about 0.02, from about 0.008 to about 0.02, from about 0.009 to about 0.02, from about 0.01 to about 0.02, from about 0.001 to about 0.01, from about 0.002 to about 0.01, from about 0.003 to about 0.01, from about 0.004 to about 0.01, from about 0.005 to about 0.01, from about 0.006 to about 0.01, from about 0.007 to about 0.01, from about 0.008 to about 0.01, or from about 0.009 to about 0.01% (w/w). In some further embodiments, the EP2 receptor agonist is present at about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, or 0.1% (w/w). In some further embodiments, the EP2 receptor agonist is present in an amount of about 0.001% w/w. The numerical values above represent amounts of the active ingredient in % (w/w).

In some embodiments, the EP2 receptor agonist is a compound of Formula

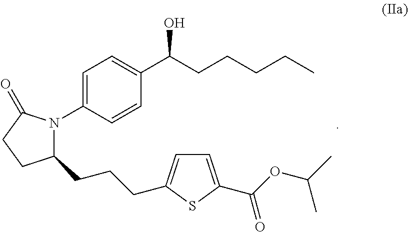

In other embodiments, the EP2 receptors agonist is any appropriate pharmaceutical salt, prodrug and/or analog of the compound of Formula (IIa). In some further embodiment, the EP2 receptor is present in an amount approximately equal to or less than about 0.05% w/w. In some further embodiments, the EP2 receptor agonist is present from about 0.0002 to about 0.05, from about 0.0004 to about 0.05, from about 0.0006 to about 0.05, from about 0.0008 to about 0.05, from about 0.001 to about 0.05, from about 0.002 to about 0.05, from about 0.004 to about 0.05, from about 0.006 to about 0.05, from about 0.008 to about 0.05, from about 0.01 to about 0.05, from about 0.02 to about 0.05, from about 0.03 to about 0.05, from about 0.03 to about 0.05, from about 0.0002 to about 0.04, from about 0.0004 to about 0.04, from about 0.0006 to about 0.04, from about 0.0008 to about 0.04, from about 0.001 to about 0.04, from about 0.002 to about 0.04, from about 0.004 to about 0.04, from about 0.006 to about 0.04, from about 0.008 to about 0.04, from about 0.01 to about 0.04, from about 0.02 to about 0.04, from about 0.03 to about 0.04, from about 0.0002 to about 0.03, from about 0.0004 to about 0.03, from about 0.0006 to about 0.03, from about 0.0008 to about 0.03, from about 0.001 to about 0.03, from about 0.002 to about 0.03, from about 0.004 to about 0.03, from about 0.006 to about 0.03, from about 0.008 to about 0.03, from about 0.01 to about 0.03, from about 0.02 to about 0.03, from about 0.0002 to about 0.02, from about 0.0004 to about 0.02, from about 0.0006 to about 0.02, from about 0.0008 to about 0.02, from about 0.001 to about 0.02, from about 0.002 to about 0.02, from about 0.004 to about 0.02, from about 0.006 to about 0.02, from about 0.008 to about 0.02, from about 0.01 to about 0.02, from about 0.0002 to about 0.01, from about 0.0004 to about 0.01, from about 0.0006 to about 0.01, from about 0.0008 to about 0.01, from about 0.001 to about 0.01, from about 0.002 to about 0.01, from about 0.004 to about 0.01, from about 0.006 to about 0.01, from about 0.008 to about 0.01, from about 0.0002 to about 0.008, from about 0.0004 to about 0.008, from about 0.0006 to about 0.008, from about 0.0008 to about 0.008, from about 0.001 to about 0.008, from about 0.002 to about 0.008, from about 0.004 to about 0.008, from about 0.006 to about 0.008, from about 0.0002 to about 0.006, from about 0.0004 to about 0.006, from about 0.001 to about 0.006, from about 0.002 to about 0.006, from about 0.004 to about 0.006, or from about 0.0002 to about 0.004% (w/w). In some further embodiments, the EP2 receptor agonist is present at about 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, or 0.5% (w/w). In some further embodiments, the EP2 receptor agonist is present in an amount of about 0.0002% w/w. The numerical values above represent amounts of the active ingredient in % (w/w).

In some embodiments, the EP2 receptor agonist is a compound of Formula

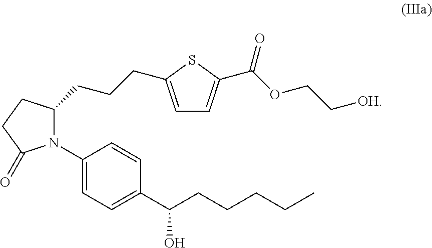

In other embodiments, the EP2 receptors agonist is any appropriate pharmaceutical salt, prodrug and/or analog of the compound of Formula (IIIa). In some further embodiments, the EP2 receptor agonist is present in an amount approximately equal to or less than about 0.1% w/w. In some further embodiments, the EP2 receptor agonist is present from about 0.001 to about 0.1, from about 0.002 to about 0.1, from about 0.003 to about 0.1, from about 0.004 to about 0.1, from about 0.005 to about 0.1, from about 0.006 to about 0.1, from about 0.007 to about 0.1, from about 0.008 to about 0.1, from about 0.009 to about 0.1, from about 0.01 to about 0.1, from about 0.02 to about 0.1, from about 0.03 to about 0.1, from about 0.04 to about 0.1, from about 0.05 to about 0.1, from about 0.06 to about 0.1, from about 0.07 to about 0.1, from about 0.08 to about 0.1, from about 0.09 to about 0.1, from about 0.001 to about 0.08, from about 0.002 to about 0.08, from about 0.003 to about 0.08, from about 0.004 to about 0.08, from about 0.005 to about 0.08, from about 0.006 to about 0.08, from about 0.007 to about 0.08, from about 0.008 to about 0.08, from about 0.009 to about 0.08, from about 0.01 to about 0.08, from about 0.02 to about 0.08, from about 0.03 to about 0.08, from about 0.04 to about 0.08, from about 0.05 to about 0.08, from about 0.06 to about 0.08, from about 0.07 to about 0.08, from about 0.001 to about 0.06, from about 0.002 to about 0.06, from about 0.003 to about 0.06, from about 0.004 to about 0.06, from about 0.005 to about 0.06, from about 0.006 to about 0.06, from about 0.007 to about 0.06, from about 0.008 to about 0.06, from about 0.009 to about 0.06, from about 0.01 to about 0.06, from about 0.02 to about 0.06, from about 0.03 to about 0.06, from about 0.04 to about 0.06, from about 0.05 to about 0.06, from about 0.001 to about 0.04, from about 0.002 to about 0.04, from about 0.003 to about 0.04, from about 0.004 to about 0.04, from about 0.005 to about 0.04, from about 0.006 to about 0.04, from about 0.007 to about 0.04, from about 0.008 to about 0.04, from about 0.009 to about 0.04, from about 0.01 to about 0.04, from about 0.02 to about 0.04, from about 0.03 to about 0.04, from about 0.001 to about 0.02, from about 0.002 to about 0.02, from about 0.003 to about 0.02, from about 0.004 to about 0.02, from about 0.005 to about 0.02, from about 0.006 to about 0.02, from about 0.007 to about 0.02, from about 0.008 to about 0.02, from about 0.009 to about 0.02, from about 0.01 to about 0.02, from about 0.001 to about 0.01, from about 0.002 to about 0.01, from about 0.003 to about 0.01, from about 0.004 to about 0.01, from about 0.005 to about 0.01, from about 0.006 to about 0.01, from about 0.007 to about 0.01, from about 0.008 to about 0.01, or from about 0.009 to about 0.01% (w/w). In some further embodiments, the EP2 receptor agonist is present at about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, or 0.1% (w/w). In some further embodiments, the EP2 receptor agonist is present in an amount of about 0.001% w/w. The numerical values above represent amounts of the active ingredient in % (w/w).

In some embodiments, the active pharmaceutical ingredient is a muscarinic receptor agonist. A muscarinic receptor agonist is an agent that enhances or increases the activity of the muscarinic acetylcholine receptor. Muscarinic receptor agonists may bind directly to the muscarinic acetylcholine receptor. Examples of a muscarinic receptor agonist include without limitation, aceclidine, arecoline, cevimeline and pilocarpine. In some embodiments, the muscarinic receptor agonist is pilocarpine. In other embodiments, the muscarinic receptor agonist is any appropriate pharmaceutical salt, prodrug and/or analog of pilocarpine. In some further embodiments, the pilocarpine is present in an amount approximately equal to or less than about 8% w/w. In some embodiments, the pilocarpine is present from about 0.01 to about 8, from about 0.05 to about 8, from about 0.1 to about 8, from about 0.5 to about 8, from about 1 to about 8, from about 1.5 to about 8, from about 2 to about 8, from about 2.5 to about 8, from about 3 to about 8, from about 3.5 to about 8, from about 4 to about 8, from about 4.5 to about 8, from about 5 to about 8, from about 5.5 to about 8, from about 6 to about 8, from about 6.5 to about 8, from about 7 to about 8, from about 7.5 to about 8, from about 0.01 to about 7.5, from about 0.05 to about 7.5, from about 0.1 to about 7.5, from about 0.5 to about 7.5, from about 1 to about 7.5, from about 1.5 to about 7.5, from about 2 to about 7.5, from about 2.5 to about 7.5, from about 3 to about 7.5, from about 3.5 to about 7.5, from about 4 to about 7.5, from about 4.5 to about 7.5, from about 5 to about 7.5, from about 5.5 to about 7.5, from about 6 to about 7.5, from about 6.5 to about 7.5, from about 7 to about 7.5, from about 0.01 to about 7, from about 0.05 to about 7, from about 0.1 to about 7, from about 0.5 to about 7, from about 1 to about 7, from about 1.5 to about 7, from about 2 to about 7, from about 2.5 to about 7, from about 3 to about 7, from about 3.5 to about 7, from about 4 to about 7, from about 4.5 to about 7, from about 5 to about 7, from about 5.5 to about 7, from about 6 to about 7, from about 6.5 to about 7, from about 0.01 to about 6.5, from about 0.05 to about 6.5, from about 0.1 to about 6.5, from about 0.5 to about 6.5, from about 1 to about 6.5, from about 1.5 to about 6.5, from about 2 to about 6.5, from about 2.5 to about 6.5, from about 3 to about 6.5, from about 3.5 to about 6.5, from about 4 to about 6.5, from about 4.5 to about 6.5, from about 5 to about 6.5, from about 5.5 to about 6.5, or from about 6 to about 6.5% w/w. The numerical values above represent amounts of the active ingredient in % (w/w).

In some embodiments, the pilocarpine is present from about 0.01 to about 6, from about 0.05 to about 6, from about 0.1 to about 6, from about 0.5 to about 6, from about 1 to about 6, from about 1.5 to about 6, from about 2 to about 6, from about 2.5 to about 6, from about 3 to about 6, from about 3.5 to about 6, from about 4 to about 6, from about 4.5 to about 6, from about 5 to about 6, from about 5.5 to about 6, from about 0.01 to about 5.5, from about 0.05 to about 5.5, from about 0.1 to about 5.5, from about 0.5 to about 5.5, from about 1 to about 5.5, from about 1.5 to about 5.5, from about 2 to about 5.5, from about 2.5 to about 5.5, from about 3 to about 5.5, from about 3.5 to about 5.5, from about 4 to about 5.5, from about 4.5 to about 5.5, from about 5 to about 5.5, from about 0.01 to about 5, from about 0.05 to about 5, from about 0.1 to about 5, from about 0.5 to about 5, from about 1 to about 5, from about 1.5 to about 5, from about 2 to about 5, from about 2.5 to about 5, from about 3 to about 5, from about 3.5 to about 5, or from about 4 to about 5% w/w. The numerical values above represent amounts of the active ingredient in % (w/w).

In some embodiments, the pilocarpine is present from about 4.5 to about 5, from about 0.01 to about 4.5, from about 0.05 to about 4.5, from about 0.1 to about 4.5, from about 0.5 to about 4.5, from about 1 to about 4.5, from about 1.5 to about 4.5, from about 2 to about 4.5, from about 2.5 to about 4.5, from about 3 to about 4.5, from about 3.5 to about 4.5, from about 4 to about 4.5, from about 0.01 to about 4, from about 0.05 to about 4, from about 0.1 to about 4, from about 0.5 to about 4, from about 1 to about 4, from about 1.5 to about 4, from about 2 to about 4, from about 2.5 to about 4, from about 3 to about 4, from about 3.5 to about 4, from about 0.01 to about 3.5, from about 0.05 to about 3.5, from about 0.1 to about 3.5, from about 0.5 to about 3.5, from about 1 to about 3.5, from about 1.5 to about 3.5, from about 2 to about 3.5, from about 2.5 to about 3.5, from about 3 to about 3.5, from about 0.01 to about 3, from about 0.05 to about 3, from about 0.1 to about 3, from about 0.5 to about 3, from about 1 to about 3, from about 1.5 to about 3, from about 2 to about 3, from about 2.5 to about 3, from about 0.01 to about 2.5, from about 0.05 to about 2.5, from about 0.1 to about 2.5, from about 0.5 to about 2.5, from about 1 to about 2.5, from about 1.5 to about 2.5, from about 2 to about 2.5, from about 0.01 to about 2, from about 0.05 to about 2, from about 0.1 to about 2, from about 0.5 to about 2, from about 1 to about 2, from about 1.5 to about 2, from about 0.01 to about 1.5, from about 0.05 to about 1.5, from about 0.1 to about 1.5, from about 0.5 to about 1.5, from about 1 to about 1.5, from about 0.01 to about 1, from about 0.05 to about 1, from about 0.1 to about 1, from about 0.5 to about 1, from about 0.01 to about 0.5, from about 0.05 to about 0.5, from about 0.1 to about 0.5, from about 0.01 to about 0.1, from about 0.05 to about 0.1, or from about 0.01 to about 0.05% w/w. In some embodiments, the pilocarpine is present at about 0.01, 0.05, 0.1, 0.5, 1, 1.5, 2, 2.5, 3, 3.5, 4, 4.5, 5, 5.5, 6, 6.5, 7, 7.5, or 8% (w/w). In some embodiments, the pilocarpine is present in an amount of about 0.01% w/w. The numerical values above represent amounts of the active ingredient in % (w/w).

In some embodiments, the active pharmaceutical ingredient is a prostaglandin analog. A prostaglandin analog is a compound, agent or molecule capable of binding a prostaglandin receptor. The structure of a prostaglandin analog may be similar to a natural prostaglandin. Examples of prostaglandin analogs include without limitation, bimatoprost, latanoprost, and travoprost. Additional examples include any pharmaceutical salts, any prodrugs and/or any functional analogs of bimatoprost, travoprost and latanoprost. In some embodiments, the prostaglandin analog is bimatoprost. Bimatoprost refers, in the customary sense, to CAS Registry No. 155206-00-1. In other embodiments, the prostaglandin analog is any appropriate pharmaceutical salt, prodrug and/or analog of the compound of bimatoprost. In some embodiments, bimatoprost is present in an amount approximately equal to or less than about 0.1% w/w. In some embodiments, bimatoprost is present from about 0.001 to about 0.1, from about 0.002 to about 0.1, from about 0.003 to about 0.1, from about 0.004 to about 0.1, from about 0.005 to about 0.1, from about 0.006 to about 0.1, from about 0.007 to about 0.1, from about 0.008 to about 0.1, from about 0.009 to about 0.1, from about 0.01 to about 0.1, from about 0.02 to about 0.1, from about 0.03 to about 0.1, from about 0.04 to about 0.1, from about 0.05 to about 0.1, from about 0.06 to about 0.1, from about 0.07 to about 0.1, from about 0.08 to about 0.1, from about 0.09 to about 0.1, from about 0.001 to about 0.08, from about 0.002 to about 0.08, from about 0.003 to about 0.08, from about 0.004 to about 0.08, from about 0.005 to about 0.08, from about 0.006 to about 0.08, from about 0.007 to about 0.08, from about 0.008 to about 0.08, from about 0.009 to about 0.08, from about 0.01 to about 0.08, from about 0.02 to about 0.08, from about 0.03 to about 0.08, from about 0.04 to about 0.08, from about 0.05 to about 0.08, from about 0.06 to about 0.08, or from about 0.07 to about 0.08% w/w. The numerical values above represent amounts of the active ingredient in % (w/w).

In some embodiments, bimatoprost is present from about 0.001 to about 0.06, from about 0.002 to about 0.06, from about 0.003 to about 0.06, from about 0.004 to about 0.06, from about 0.005 to about 0.06, from about 0.006 to about 0.06, from about 0.007 to about 0.06, from about 0.008 to about 0.06, from about 0.009 to about 0.06, from about 0.01 to about 0.06, from about 0.02 to about 0.06, from about 0.03 to about 0.06, from about 0.04 to about 0.06, from about 0.05 to about 0.06, from about 0.001 to about 0.04, from about 0.002 to about 0.04, from about 0.003 to about 0.04, from about 0.004 to about 0.04, from about 0.005 to about 0.04, from about 0.006 to about 0.04, from about 0.007 to about 0.04, from about 0.008 to about 0.04, from about 0.009 to about 0.04, from about 0.01 to about 0.04, from about 0.02 to about 0.04, from about 0.03 to about 0.04, from about 0.001 to about 0.02, from about 0.002 to about 0.02, from about 0.003 to about 0.02, from about 0.004 to about 0.02, from about 0.005 to about 0.02, from about 0.006 to about 0.02, from about 0.007 to about 0.02, from about 0.008 to about 0.02, from about 0.009 to about 0.02, from about 0.01 to about 0.02, from about 0.001 to about 0.01, from about 0.002 to about 0.01, from about 0.003 to about 0.01, from about 0.004 to about 0.01, from about 0.005 to about 0.01, from about 0.006 to about 0.01, from about 0.007 to about 0.01, from about 0.008 to about 0.01, or from about 0.009 to about 0.01% (w/w). In some embodiments, bimatoprost is present at about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, or 0.1% (w/w). In some embodiments, bimatoprost is present in an amount of about 0.001% w/w. The numerical values above represent amounts of the active ingredient in % (w/w).

In other embodiments, the prostaglandin analog is latanoprost. Latanoprost refers, in the customary sense, to CAS Registry No. 130209-82-4. In other embodiments, the prostaglandin analog is any appropriate pharmaceutical salt, prodrug and/or analog of the compound of latanoprost. In some embodiments, the latanoprost is present in an amount approximately equal to or less than about 0.1% w/w. In some embodiments, latanoprost is present from about 0.0003 to about 0.1, from about 0.0005 to about 0.1, from about 0.0007 to about 0.1, from about 0.0009 to about 0.1, from about 0.001 to about 0.1, from about 0.003 to about 0.1, from about 0.005 to about 0.1, from about 0.007 to about 0.1, from about 0.009 to about 0.1, from about 0.01 to about 0.1, from about 0.03 to about 0.1, from about 0.05 to about 0.1, from about 0.07 to about 0.1, from about 0.09 to about 0.1, from about 0.0003 to about 0.09, from about 0.0005 to about 0.09, from about 0.0007 to about 0.09, from about 0.0009 to about 0.09, from about 0.001 to about 0.09, from about 0.003 to about 0.09, from about 0.005 to about 0.09, from about 0.007 to about 0.09, from about 0.009 to about 0.09, from about 0.01 to about 0.09, from about 0.03 to about 0.09, from about 0.05 to about 0.09, from about 0.07 to about 0.09, from about 0.0003 to about 0.07, from about 0.0005 to about 0.07, from about 0.0007 to about 0.07, from about 0.0009 to about 0.07, from about 0.001 to about 0.07, from about 0.003 to about 0.07, from about 0.005 to about 0.07, from about 0.007 to about 0.07, from about 0.009 to about 0.07, from about 0.01 to about 0.07, from about 0.03 to about 0.07, from about 0.05 to about 0.07, from about 0.0003 to about 0.05, from about 0.0005 to about 0.05, from about 0.0007 to about 0.05, from about 0.0009 to about 0.05, from about 0.001 to about 0.05, from about 0.003 to about 0.05, from about 0.005 to about 0.05, from about 0.007 to about 0.05, from about 0.009 to about 0.05, from about 0.01 to about 0.05, or from about 0.03 to about 0.05% w/w. The numerical values above represent amounts of the active ingredient in % (w/w).

In some embodiments, latanoprost is present from about 0.0003 to about 0.03, from about 0.0005 to about 0.03, from about 0.0007 to about 0.03, from about 0.0009 to about 0.03, from about 0.001 to about 0.03, from about 0.003 to about 0.03, from about 0.005 to about 0.03, from about 0.007 to about 0.03, from about 0.009 to about 0.03, from about 0.01 to about 0.03, from about 0.0003 to about 0.01, from about 0.0005 to about 0.01, from about 0.0007 to about 0.01, from about 0.0009 to about 0.01, from about 0.001 to about 0.01, from about 0.003 to about 0.01, from about 0.005 to about 0.01, from about 0.007 to about 0.01, from about 0.009 to about 0.01, from about 0.0003 to about 0.009, from about 0.0005 to about 0.009, from about 0.0007 to about 0.009, from about 0.0009 to about 0.009, from about 0.001 to about 0.009, from about 0.003 to about 0.009, from about 0.005 to about 0.009, from about 0.007 to about 0.009, from about 0.0003 to about 0.007, from about 0.0005 to about 0.007, from about 0.0007 to about 0.007, from about 0.0009 to about 0.007, from about 0.001 to about 0.007, from about 0.003 to about 0.007, from about 0.005 to about 0.007, from about 0.0003 to about 0.005, from about 0.0005 to about 0.005, from about 0.0007 to about 0.005, from about 0.0009 to about 0.005, from about 0.001 to about 0.005, from about 0.003 to about 0.005, from about 0.0003 to about 0.003, from about 0.0005 to about 0.003, from about 0.0007 to about 0.003, from about 0.0009 to about 0.003, from about 0.001 to about 0.003, from about 0.0003 to about 0.001, from about 0.0005 to about 0.001, from about 0.0007 to about 0.001, from about 0.0009 to about 0.001, from about 0.0003 to about 0.0009, from about 0.0005 to about 0.0009, from about 0.0007 to about 0.0009, from about 0.0003 to about 0.0007, from about 0.0005 to about 0.0007, or from about 0.0003 to about 0.0005% (w/w). In some embodiments, the latanoprost is present at about 0.1, 0.09, 0.07, 0.05, 0.03, 0.01, 0.009, 0.007, 0.005, 0.003, 0.001, 0.0009, 0.0007, 0.0005, or 0.0003% (w/w). In some embodiments, the latanoprost is present in an amount of about 0.0003% w/w. The numerical values above represent amounts of the active ingredient in % (w/w).

In some embodiments, the prostaglandin analog is travoprost. Travoprost refers, in the customary sense, to CAS Registry No. 157283-68-6. In other embodiments, the prostaglandin analog is any appropriate pharmaceutical salt, prodrug and/or analog of the compound of travoprost. In some embodiments, the travoprost is present in an amount approximately equal to or less than about 0.1% w/w. In some embodiments, the travoprost is present in an amount from about 0.0002 to about 0.1, from about 0.0004 to about 0.1, from about 0.0006 to about 0.1, from about 0.0008 to about 0.1, from about 0.001 to about 0.1, from about 0.002 to about 0.1, from about 0.004 to about 0.1, from about 0.006 to about 0.1, from about 0.008 to about 0.1, from about 0.01 to about 0.1, from about 0.02 to about 0.1, from about 0.04 to about 0.1, from about 0.06 to about 0.1, from about 0.08 to about 0.1, from about 0.0002 to about 0.08, from about 0.0004 to about 0.08, from about 0.0006 to about 0.08, from about 0.0008 to about 0.08, from about 0.001 to about 0.08, from about 0.002 to about 0.08, from about 0.004 to about 0.08, from about 0.006 to about 0.08, from about 0.008 to about 0.08, from about 0.01 to about 0.08, from about 0.02 to about 0.08, from about 0.04 to about 0.08, from about 0.06 to about 0.08, from about 0.0002 to about 0.06, from about 0.0004 to about 0.06, from about 0.0006 to about 0.06, from about 0.0008 to about 0.06, from about 0.001 to about 0.06, from about 0.002 to about 0.06, from about 0.004 to about 0.06, from about 0.006 to about 0.06, from about 0.008 to about 0.06, from about 0.01 to about 0.06, from about 0.02 to about 0.06, or from about 0.04 to about 0.06% w/w. The numerical values above represent amounts of the active ingredient in % (w/w).

In some embodiments, the travoprost is present in an amount from about 0.0002 to about 0.04, from about 0.0004 to about 0.04, from about 0.0006 to about 0.04, from about 0.0008 to about 0.04, from about 0.001 to about 0.04, from about 0.002 to about 0.04, from about 0.004 to about 0.04, from about 0.006 to about 0.04, from about 0.008 to about 0.04, from about 0.01 to about 0.04, from about 0.02 to about 0.04, from about 0.0002 to about 0.02, from about 0.0004 to about 0.02, from about 0.0006 to about 0.02, from about 0.0008 to about 0.02, from about 0.001 to about 0.02, from about 0.002 to about 0.02, from about 0.004 to about 0.02, from about 0.006 to about 0.02, from about 0.008 to about 0.02, from about 0.01 to about 0.02, from about 0.0002 to about 0.01, from about 0.0004 to about 0.01, from about 0.0006 to about 0.01, from about 0.0008 to about 0.01, from about 0.001 to about 0.01, from about 0.002 to about 0.01, from about 0.004 to about 0.01, from about 0.006 to about 0.01, from about 0.008 to about 0.01, from about 0.0002 to about 0.008, from about 0.0004 to about 0.008, from about 0.0006 to about 0.008, from about 0.0008 to about 0.008, from about 0.001 to about 0.008, from about 0.002 to about 0.008, from about 0.004 to about 0.008, from about 0.006 to about 0.008, from about 0.0002 to about 0.006, from about 0.0004 to about 0.006, from about 0.0006 to about 0.006, from about 0.0008 to about 0.006, from about 0.001 to about 0.006, from about 0.002 to about 0.006, from about 0.004 to about 0.006, from about 0.0002 to about 0.004, from about 0.0004 to about 0.004, from about 0.0006 to about 0.004, from about 0.0008 to about 0.004, from about 0.001 to about 0.004, from about 0.002 to about 0.004, from about 0.0002 to about 0.002, from about 0.0004 to about 0.002, from about 0.0006 to about 0.002, from about 0.0008 to about 0.002, from about 0.001 to about 0.002, from about 0.0002 to about 0.001, from about 0.0004 to about 0.001, from about 0.0006 to about 0.001, from about 0.0008 to about 0.001, from about 0.0002 to about 0.0008, from about 0.0004 to about 0.0008, from about 0.0006 to about 0.0008, from about 0.0002 to about 0.0006, from about 0.0004 to about 0.0006, or from about 0.0002 to about 0.0004% (w/w). In some embodiments, the travoprost is present at about 0.1, 0.08, 0.06, 0.04, 0.02, 0.01, 0.008, 0.006, 0.004, 0.002, 0.001, 0.0008, 0.0006, 0.0004, or 0.0002% (w/w). In some embodiments, travoprost is present in an amount of about 0.0002% w/w. The numerical values above represent amounts of the active ingredient in % (w/w).

As mentioned above the non-aqueous composition provided herein may include a vasoconstrictor agent. A vasoconstrictor agent is an agent having a vasoconstriction effect on blood vessel within an organism (e.g. a mammal such as a human). Vasoconstriction typically results from the narrowing of blood vessels resulting from contraction of the muscular wall of the vessels. Vasoconstriction may be a mechanism by which the body regulates and maintains mean arterial pressure. Therefore, vasoconstrictors or vasoconstrictor agents are often agents causing a general increase in systemic blood pressure, but at the same time may cause a localized reduction in blood flow. In some embodiments, the vasoconstrictor agent is an alpha adrenergic agonist. An alpha adrenergic agonist is an agent (e.g., drug, compound), which stimulates (e.g. selectively stimulates) alpha adrenergic receptors. Alpha adrenergic receptors are G protein-coupled receptors that may be bound by noradrenalin and adrenaline. In some embodiments, binding of an agonist to an alpha adrenergic receptor leads to vasoconstriction, which causes a sympathetic response, where the heart rate increases, the pupils dilate and blood flow is being diverted from non-essential organs to the skeletal muscle. A non-limiting example of an alpha adrenergic agonist is brimonidine. In some embodiments, the alpha adrenergic agonist is brimonidine. Brimonidine refers, in the customary sense, to CAS Registry No. 59803-98-4. In other embodiments, the alpha adrenergic agonist is any appropriate pharmaceutical salt, prodrug and/or analog of the compound of brimonidine. In some embodiments, the brimonidine is present in an amount approximately equal to or less than 1% w/w. In some embodiments, the brimonidine is present from about 0.001 to about 0.1, from about 0.002 to about 0.1, from about 0.003 to about 0.1, from about 0.004 to about 0.1, from about 0.005 to about 0.1, from about 0.006 to about 0.1, from about 0.007 to about 0.1, from about 0.008 to about 0.1, from about 0.009 to about 0.1, from about 0.01 to about 0.1, from about 0.02 to about 0.1, from about 0.03 to about 0.1, from about 0.04 to about 0.1, from about 0.05 to about 0.1, from about 0.06 to about 0.1, from about 0.07 to about 0.1, from about 0.08 to about 0.1, from about 0.09 to about 0.1, from about 0.001 to about 0.08, from about 0.002 to about 0.08, from about 0.003 to about 0.08, from about 0.004 to about 0.08, from about 0.005 to about 0.08, from about 0.006 to about 0.08, from about 0.007 to about 0.08, from about 0.008 to about 0.08, from about 0.009 to about 0.08, from about 0.01 to about 0.08, from about 0.02 to about 0.08, from about 0.03 to about 0.08, from about 0.04 to about 0.08, from about 0.05 to about 0.08, from about 0.06 to about 0.08, or from about 0.07 to about 0.08% w/w. The numerical values above represent amounts of the active ingredient in % (w/w).

In some embodiments, the brimonidine is present from about 0.001 to about 0.06, from about 0.002 to about 0.06, from about 0.003 to about 0.06, from about 0.004 to about 0.06, from about 0.005 to about 0.06, from about 0.006 to about 0.06, from about 0.007 to about 0.06, from about 0.008 to about 0.06, from about 0.009 to about 0.06, from about 0.01 to about 0.06, from about 0.02 to about 0.06, from about 0.03 to about 0.06, from about 0.04 to about 0.06, from about 0.05 to about 0.06, from about 0.001 to about 0.04, from about 0.002 to about 0.04, from about 0.003 to about 0.04, from about 0.004 to about 0.04, from about 0.005 to about 0.04, from about 0.006 to about 0.04, from about 0.007 to about 0.04, from about 0.008 to about 0.04, from about 0.009 to about 0.04, from about 0.01 to about 0.04, from about 0.02 to about 0.04, from about 0.03 to about 0.04, from about 0.001 to about 0.02, from about 0.002 to about 0.02, from about 0.003 to about 0.02, from about 0.004 to about 0.02, from about 0.005 to about 0.02, from about 0.006 to about 0.02, from about 0.007 to about 0.02, from about 0.008 to about 0.02, from about 0.009 to about 0.02, from about 0.01 to about 0.02, from about 0.001 to about 0.01, from about 0.002 to about 0.01, from about 0.003 to about 0.01, from about 0.004 to about 0.01, from about 0.005 to about 0.01, from about 0.006 to about 0.01, from about 0.007 to about 0.01, from about 0.008 to about 0.01, or from about 0.009 to about 0.01% (w/w). In some embodiments, the brimonidine is present at about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, or 0.1% (w/w). In some embodiments, the brimonidine is present in an amount of about 0.001% w/w. The numerical values above represent amounts of the active ingredient in % (w/w).

In some embodiments, the alpha adrenergic agonist is an alpha adrenergic agonist compound. In some embodiments, the alpha adrenergic agonist compound has the Formula

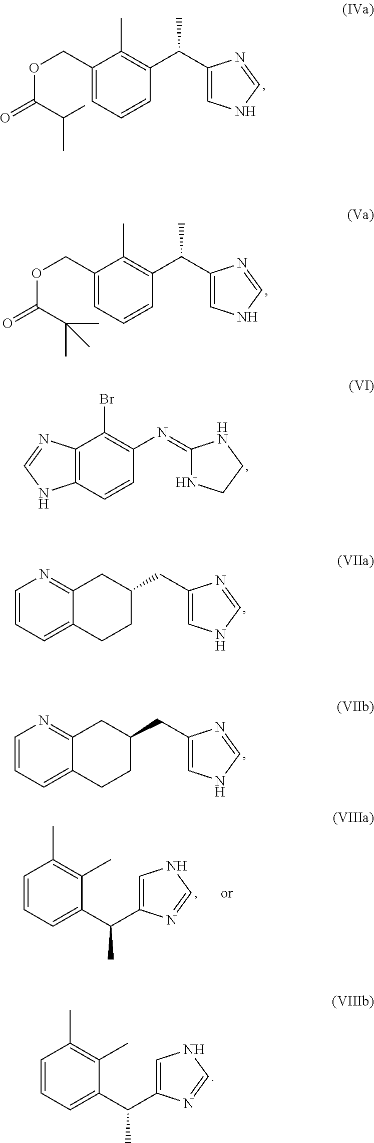

In some embodiments, the alpha adrenergic agonist compound has the Formula

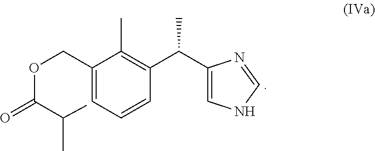

In some embodiments, the alpha adrenergic agonist compound has the Formula

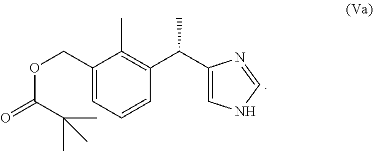

In some embodiments, the alpha adrenergic agonist compound has the Formula

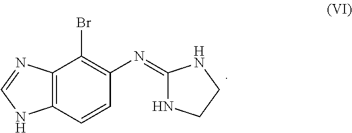

In some embodiments, the alpha adrenergic agonist compound has the Formula

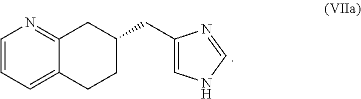

In some embodiments, the alpha adrenergic agonist compound has the Formula

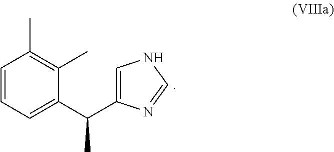

In other embodiments, the alpha adrenergic agonist is any appropriate pharmaceutical salt, prodrug and/or analog of the compound of Formula (IVa), (Va), (VI), (VIIa), (VIIb), (VIIIa), or (VIIIb). In other embodiments, the alpha adrenergic agonist is any appropriate pharmaceutical salt, prodrug and/or analog of the compound of Formula (IVa), (Va), (VI), (VIIa), or (VIIIa).

In some embodiments, the alpha adrenergic agonist compound is present in an amount approximately equal to or less than 1% w/w. In some embodiments, the alpha adrenergic agonist compound is present from about 0.001 to about 0.1, from about 0.002 to about 0.1, from about 0.003 to about 0.1, from about 0.004 to about 0.1, from about 0.005 to about 0.1, from about 0.006 to about 0.1, from about 0.007 to about 0.1, from about 0.008 to about 0.1, from about 0.009 to about 0.1, from about 0.01 to about 0.1, from about 0.02 to about 0.1, from about 0.03 to about 0.1, from about 0.04 to about 0.1, from about 0.05 to about 0.1, from about 0.06 to about 0.1, from about 0.07 to about 0.1, from about 0.08 to about 0.1, from about 0.09 to about 0.1, from about 0.001 to about 0.08, from about 0.002 to about 0.08, from about 0.003 to about 0.08, from about 0.004 to about 0.08, from about 0.005 to about 0.08, from about 0.006 to about 0.08, from about 0.007 to about 0.08, from about 0.008 to about 0.08, from about 0.009 to about 0.08, from about 0.01 to about 0.08, from about 0.02 to about 0.08, from about 0.03 to about 0.08, from about 0.04 to about 0.08, from about 0.05 to about 0.08, from about 0.06 to about 0.08, or from about 0.07 to about 0.08% w/w. The numerical values above represent amounts of the active ingredient in % (w/w).

In some embodiments, the alpha adrenergic agonist compound is present from about 0.001 to about 0.06, from about 0.002 to about 0.06, from about 0.003 to about 0.06, from about 0.004 to about 0.06, from about 0.005 to about 0.06, from about 0.006 to about 0.06, from about 0.007 to about 0.06, from about 0.008 to about 0.06, from about 0.009 to about 0.06, from about 0.01 to about 0.06, from about 0.02 to about 0.06, from about 0.03 to about 0.06, from about 0.04 to about 0.06, from about 0.05 to about 0.06, from about 0.001 to about 0.04, from about 0.002 to about 0.04, from about 0.003 to about 0.04, from about 0.004 to about 0.04, from about 0.005 to about 0.04, from about 0.006 to about 0.04, from about 0.007 to about 0.04, from about 0.008 to about 0.04, from about 0.009 to about 0.04, from about 0.01 to about 0.04, from about 0.02 to about 0.04, from about 0.03 to about 0.04, from about 0.001 to about 0.02, from about 0.002 to about 0.02, from about 0.003 to about 0.02, from about 0.004 to about 0.02, from about 0.005 to about 0.02, from about 0.006 to about 0.02, from about 0.007 to about 0.02, from about 0.008 to about 0.02, from about 0.009 to about 0.02, from about 0.01 to about 0.02, from about 0.001 to about 0.01, from about 0.002 to about 0.01, from about 0.003 to about 0.01, from about 0.004 to about 0.01, from about 0.005 to about 0.01, from about 0.006 to about 0.01, from about 0.007 to about 0.01, from about 0.008 to about 0.01, or from about 0.009 to about 0.01% (w/w). In some embodiments, the alpha adrenergic agonist compound is present at about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, or 0.1% (w/w). In some embodiments, the alpha adrenergic agonist compound is present in an amount of about 0.001% w/w. The numerical values above represent amounts of the active ingredient in % (w/w).

In other embodiments, the vasoconstrictor agent is a beta adrenergic antagonist. A beta adrenergic antagonist is an agent (e.g., drug, compound), which inhibits (e.g. decreases) the stimulation of beta adrenergic receptors. Stimulation of beta adrenergic receptors induces smooth muscle relaxation, whereas blocking beta adrenergic receptors typically causes contraction of smooth muscles. Therefore, beta adrenergic antagonists may cause vasoconstriction. Examples of beta adrenergic antagonists include without limitation befunolol, betaxolol, carteolol, levobunolol, metipranolol, timolol, and mepindolol.

In some embodiments, the beta adrenergic antagonist is timolol. In some embodiments, the timolol is timolol maleate. Timolol maleate refers, in the customary sense, to CAS Registry No. 26839-75-8. The chemical name of timolol maleate is (−)-1-tert-butylamino-3-[(4-morpholino-1,2,5-thiodiazol-3-yl)oxy]-2-propanol maleate. Timolol maleate has a molecular weight of 432.50 g/mol and is commercially available from Merck as TIMOPTIC®. In other embodiments, the timolol is timolol hemihydrate. In other embodiments, the beta adrenergic antagonist is any appropriate pharmaceutical salt, prodrug and/or analog of timolol. In some embodiments, the timolol is present in an amount approximately equal to or less than about 0.5% w/w. In some embodiments, the timolol is present from about 0.01 to about 1, from about 0.02 to about 1, from about 0.03 to about 1, from about 0.04 to about 1, from about 0.05 to about 1, from about 0.06 to about 1, from about 0.07 to about 1, from about 0.08 to about 1, from about 0.09 to about 1, from about 0.1 to about 1, from about 0.2 to about 1, from about 0.3 to about 1, from about 0.4 to about 1, from about 0.5 to about 1, from about 0.6 to about 1, from about 0.7 to about 1, from about 0.8 to about 1, from about 0.9 to about 1, from about 0.01 to about 0.9, from about 0.02 to about 0.9, from about 0.03 to about 0.9, from about 0.04 to about 0.9, from about 0.05 to about 0.9, from about 0.06 to about 0.9, from about 0.07 to about 0.9, from about 0.08 to about 0.9, from about 0.09 to about 0.9, from about 0.1 to about 0.9, from about 0.2 to about 0.9, from about 0.3 to about 0.9, from about 0.4 to about 0.9, from about 0.5 to about 0.9, from about 0.6 to about 0.9, from about 0.7 to about 0.9, from about 0.8 to about 0.9, from about 0.01 to about 0.8, from about 0.02 to about 0.8, from about 0.03 to about 0.8, from about 0.04 to about 0.8, from about 0.05 to about 0.8, from about 0.06 to about 0.8, from about 0.07 to about 0.8, from about 0.08 to about 0.8, from about 0.09 to about 0.8, from about 0.1 to about 0.8, from about 0.2 to about 0.8, from about 0.3 to about 0.8, from about 0.4 to about 0.8, from about 0.5 to about 0.8, from about 0.6 to about 0.8, or from about 0.7 to about 0.8% w/w. The numerical values above represent amounts of the active ingredient in % (w/w).

In some embodiments, the timolol is present from about 0.01 to about 0.7, from about 0.02 to about 0.7, from about 0.03 to about 0.7, from about 0.04 to about 0.7, from about 0.05 to about 0.7, from about 0.06 to about 0.7, from about 0.07 to about 0.7, from about 0.08 to about 0.7, from about 0.09 to about 0.7, from about 0.1 to about 0.7, from about 0.2 to about 0.7, from about 0.3 to about 0.7, from about 0.4 to about 0.7, from about 0.5 to about 0.7, from about 0.6 to about 0.7, from about 0.01 to about 0.6, from about 0.02 to about 0.6, from about 0.03 to about 0.6, from about 0.04 to about 0.6, from about 0.05 to about 0.6, from about 0.06 to about 0.6, from about 0.07 to about 0.6, from about 0.08 to about 0.6, from about 0.09 to about 0.6, from about 0.1 to about 0.6, from about 0.2 to about 0.6, from about 0.3 to about 0.6, from about 0.4 to about 0.6, from about 0.5 to about 0.6, from about 0.01 to about 0.5, from about 0.02 to about 0.5, from about 0.03 to about 0.5, from about 0.04 to about 0.5, from about 0.05 to about 0.5, from about 0.06 to about 0.5, from about 0.07 to about 0.5, from about 0.08 to about 0.5, from about 0.09 to about 0.5, from about 0.1 to about 0.5, from about 0.2 to about 0.5, from about 0.3 to about 0.5, from about 0.4 to about 0.5, from about 0.01 to about 0.4, from about 0.02 to about 0.4, from about 0.03 to about 0.4, from about 0.04 to about 0.4, from about 0.05 to about 0.4, from about 0.06 to about 0.4, from about 0.07 to about 0.4, from about 0.08 to about 0.4, from about 0.09 to about 0.4, from about 0.1 to about 0.4, from about 0.2 to about 0.4, from about 0.3 to about 0.4, from about 0.01 to about 0.3, from about 0.02 to about 0.3, from about 0.03 to about 0.3, from about 0.04 to about 0.3, from about 0.05 to about 0.3, from about 0.06 to about 0.3, from about 0.07 to about 0.3, from about 0.08 to about 0.3, from about 0.09 to about 0.3, from about 0.1 to about 0.3, or from about 0.2 to about 0.3% w/w. The numerical values above represent amounts of the active ingredient in % (w/w).

In some embodiments, the timolol is present from about 0.01 to about 0.2, from about 0.02 to about 0.2, from about 0.03 to about 0.2, from about 0.04 to about 0.2, from about 0.05 to about 0.2, from about 0.06 to about 0.2, from about 0.07 to about 0.2, from about 0.08 to about 0.2, from about 0.09 to about 0.2, from about 0.1 to about 0.2, from about 0.01 to about 0.1, from about 0.02 to about 0.1, from about 0.03 to about 0.1, from about 0.04 to about 0.1, from about 0.05 to about 0.1, from about 0.06 to about 0.1, from about 0.07 to about 0.1, from about 0.08 to about 0.1, from about 0.09 to about 0.1, from about 0.01 to about 0.09, from about 0.02 to about 0.09, from about 0.03 to about 0.09, from about 0.04 to about 0.09, from about 0.05 to about 0.09, from about 0.06 to about 0.09, from about 0.07 to about 0.09, from about 0.08 to about 0.09, from about 0.01 to about 0.08, from about 0.02 to about 0.08, from about 0.03 to about 0.08, from about 0.04 to about 0.08, from about 0.05 to about 0.08, from about 0.06 to about 0.08, from about 0.07 to about 0.08, from about 0.01 to about 0.07, from about 0.02 to about 0.07, from about 0.03 to about 0.07, from about 0.04 to about 0.07, from about 0.05 to about 0.07, from about 0.06 to about 0.07, from about 0.01 to about 0.06, from about 0.02 to about 0.06, from about 0.03 to about 0.06, from about 0.04 to about 0.06, from about 0.05 to about 0.06, from about 0.01 to about 0.05, from about 0.02 to about 0.05, from about 0.03 to about 0.05, from about 0.04 to about 0.05, from about 0.01 to about 0.04, from about 0.02 to about 0.04, from about 0.03 to about 0.04, from about 0.01 to about 0.03, from about 0.02 to about 0.03, or from about 0.01 to about 0.02% w/w. In some embodiments, the timolol is present at about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9 or 1% w/w. In some embodiments, the timolol is present in amount of about 0.05% w/w. The numerical values above represent amounts of the active ingredient in % (w/w).

The active pharmaceutical ingredient provided herein may be an anti-infective agent. An anti-infective agent is an agent capable of inhibiting (e.g. reducing) growth, spreading of or killing of bacterial, fungal or viral organisms. Examples of anti-infective agents include antibacterial, antibiotic, antifungal, antiprotozoan, and antiviral agents. Thus, in some embodiments, the active pharmaceutical ingredient is an anti-infective agent. In some embodiments, the anti-infective agent is gatifloxacin. Gatifloxacin refers, in the customary sense, to CAS Registry No. 112811-59-3. The chemical name of gatifloxacin is 1-cyclopropyl-6-fluoro-8-methoxy-7-(3-methylpiperazin-1-yl)-4-oxo-quinoline-3-carboxylic acid. In other embodiments, the anti-infective agent is any appropriate pharmaceutical salt, prodrug and/or analog of gatifloxacin. In some embodiments, gatifloxacin is present in an amount approximately equal to or less than about 1% w/w. In some embodiments, the gatifloxacin is present from about 0.01 to about 3, from about 0.05 to about 3, from about 0.1 to about 3, from about 0.5 to about 3, from about 1 to about 3, from about 1.5 to about 3, from about 2 to about 3, from about 2.5 to about 3, from about 0.01 to about 2.5, from about 0.05 to about 2.5, from about 0.1 to about 2.5, from about 0.5 to about 2.5, from about 1 to about 2.5, from about 1.5 to about 2.5, from about 2 to about 2.5, from about 0.01 to about 2, from about 0.05 to about 2, from about 0.1 to about 2, from about 0.5 to about 2, from about 1 to about 2, from about 1.5 to about 2, from about 0.01 to about 1.5, from about 0.05 to about 1.5, from about 0.1 to about 1.5, from about 0.5 to about 1.5, from about 1 to about 1.5, from about 0.01 to about 1, from about 0.05 to about 1, from about 0.1 to about 1, from about 0.5 to about 1, from about 0.01 to about 0.5, from about 0.05 to about 0.5, from about 0.1 to about 0.5, from about 0.01 to about 0.1, from about 0.05 to about 0.1, or from about 0.01 to about 0.0.5% w/w. In some embodiments, the gatifloxacin is present at about 0.01, 0.05, 0.1, 0.5, 1, 1.5, 2, 2.5, or 3% w/w. In some embodiments, the gatifloxacin is present in an amount of about 0.1% w/w. The numerical values above represent amounts of the active ingredient in % (w/w).

The non-aqueous compositions and products according to the embodiments of the present invention comprise a silicone excipient. A silicone excipient as defined herein is a pharmaceutically acceptable silicone-based agent with which the active pharmaceutical ingredient is combined to facilitate the application. As provided herein a silicone excipient be a silicone excipient blend. A silicone excipient blend may include two or more silicone compounds, where the constituent silicone compounds form a uniform mixture of a particular character, quality, or consistency. For example, a first silicone compound and a second silicone compound forming a blend may have different viscosities. The first silicone compound may have a low viscosity and therefore be a in a fluid state, whereas the second silicone compound may have a high viscosity and therefore be in a solid (gum) state. By combining a specific amount of the first silicone compound with a specific amount of the second silicone compound a blend with a specific viscosity is generated. For example, the viscosity of a blend including an amount of a low viscosity silicone compound and an amount of a high viscosity silicone compound may have a viscosity which is higher than the viscosity of the low viscosity silicone compound and lower than the viscosity of the high viscosity silicone compound. Non-limiting examples of silicone compounds useful for the silicone excipient blends provided herein are dimethiconol, dimethicone, cyclopentasiloxane, decamethylcyclopentasiloxane, alkylmethyl siloxane copolyol, alkylmethyl siloxane and stearyltrimethylsilane.

In some embodiments, the silicone excipient is a silicone excipient blend. In some embodiments, the non-aqueous composition includes a plurality of excipient blends. For example, the non-aqueous composition may include a first silicone excipient blend, a second silicone excipient blend, a third silicone excipient blend, a fourth silicone excipient blend, a fifth silicone excipient blend, a sixth silicone excipient blend and/or a seventh silicone excipient blend. In other embodiments, the non-aqueous composition includes a first silicone excipient blend and a second silicone excipient blend. In other embodiments, the non-aqueous composition includes a first silicone excipient blend, a second silicone excipient blend and a third silicone excipient blend. In other embodiments, the non-aqueous composition includes a first silicone excipient blend, a second silicone excipient blend, a third silicone excipient blend, and a fourth silicone excipient blend. Where, the non-aqueous composition includes a plurality of excipient blends (e.g. a “first, second, third, fourth, fifth, sixth and/or seventh” silicone excipient blend), each excipient blend may be different. For example, each of the first, second, third, fourth, fifth, sixth and/or seventh silicone excipient blends, in some embodiments, are different (i.e. chemically different or having at least one different chemical component such as a silicone based chemical component). In some embodiments, the second silicone excipient blend as used herein is chemically different from the other silicone excipient blends present in the non-aqueous composition (e.g. first, third, forth, fifth, sixth or seventh silicone excipient blend). In some embodiments, a “third” silicone excipient blend is different from the other, first, second, forth, fifth, sixth, or seventh silicone excipient blend. A first, second, third, forth, fifth, sixth or seventh silicone excipient blend can be any silicone excipient blend provided herein (e.g. dimethiconol, dimethicone, cyclopentasiloxane, decamethylcyclopentasiloxane, alkylmethyl siloxane copolyol, alkylmethyl siloxane and stearyltrimethylsilane) or any silicone excipient blend suitable for the non-aqueous compositions according to the embodiments provided herein.

In some embodiments, the first silicone excipient blend includes dimethicone and dimethiconol. Dimethicone, also known in the art as polydimethylsiloxane (PDMS) is a silicon compound having the chemical formula CH₃[Si(CH₃)₂O]ₙSi(CH₃)₃, where n is the number of repeating monomer [SiO(CH₃)₂] units. Dimethicone refers, in the customary sense, to CAS Registry No. 70131-67-8. Dimethiconol is a hydroxyl-terminated polydimethylsiloxane and refers, in the customary sense, to CAS Registry No. 63148-62-9. Depending on the number of repeating methylsiloxane units, the silicone compounds dimethicone and dimethiconol may exhibit different viscosities. Where the number of methylsiloxane units in the silicone compound is high the viscosity is high and where the number of methylsiloxane units is low the viscosity is low. A non-limiting example of a silicone excipient blend including dimethicone and dimethiconol useful for the compositions provided herein is Dimethiconol Blend®20. Dimethiconol Blend®20 is a clear solution of approximately 6% of an ultra-high viscosity hydroxyl-terminated polydimethylsiloxane gum (dimethiconol) in a low viscosity (non-volatile) silicone fluid (dimethicone). In some embodiments, the first silicone excipient blend is present from about 1% w/w to about 10% w/w. In some embodiments, the first silicone excipient blend is present from about 2% w/w to about 10% w/w, from about 3% w/w to about 10% w/w, from about 4% w/w to about 10% w/w, from about 5% w/w to about 10% w/w, from about 6% w/w to about 10% w/w, from about 7% w/w to about 10% w/w, from about 8% w/w to about 10% w/w, from about 9% w/w to about 10% w/w, from about 2% w/w to about 9% w/w, from about 3% w/w to about 9% w/w, from about 4% w/w to about 9% w/w, from about 5% w/w to about 9% w/w, from about 6% w/w to about 9% w/w, from about 7% w/w to about 9% w/w, from about 8% w/w to about 9% w/w, from about 2% w/w to about 8% w/w, from about 3% w/w to about 8% w/w, from about 4% w/w to about 8% w/w, from about 5% w/w to about 8% w/w, from about 6% w/w to about 8% w/w, from about 7% w/w to about 8% w/w, from about 2% w/w to about 7% w/w, from about 3% w/w to about 7% w/w, from about 4% w/w to about 7% w/w, from about 5% w/w to about 7% w/w, from about 6% w/w to about 7% w/w, from about 2% w/w to about 6% w/w, from about 3% w/w to about 6% w/w, from about 4% w/w to about 6% w/w, from about 5% w/w to about 6% w/w, from about 2% w/w to about 5% w/w, from about 3% w/w to about 5% w/w, from about 4% w/w to about 5% w/w, from about 2% w/w to about 4% w/w, from about 3% w/w to about 4% w/w, or from about 2% w/w to about 3% w/w. In some embodiments, the first silicone excipient blend is present at about 1, 2, 3, 4, 5, 6, 7, 8, 9, or 10% (w/w). The numerical values above represent amounts of silicone excipient in % (w/w). The first silicone excipient (e.g. excipient blend) may be present in a quantity sufficient (q.s.) that the total of all components (i.e. active pharmaceutical ingredients, silicone excipient blends, and lipid excipients) present in a non-aqueous composition equals 100% w/w. For example where the total of active pharmaceutical ingredients, silicone excipient blends, and lipid excipients in a non-aqueous composition is 36.35%, the amount of the first silicone excipient is 63.65% w/w thereby resulting in a total of 100% w/w for all components present in the non-aqueous composition.

In some embodiments, the second silicone excipient blend includes cyclopentasiloxane and dimethicone cross polymer. Cyclopentasiloxane is a cyclic dimethicone including five monomer [SiO(CH₃)₂] units and is therefore also called decamethylcyclopentasiloxane. A dimethicone cross polymer is a high molecular weight silicone elastomer, where a methyl group in one or more of the monomer [SiO(CH₃)₂] units is replaced with an hydrocarbon side chain of variable length (e.g. C₈H₁₇). A non-limiting example of a silicone excipient blend including cyclopentasiloxane and dimethicone cross polymer that is useful for the compositions provided herein is Elastomer®10. Elastomer®10 is a mixture of 12% high molecular weight silicone elastomer (i.e. dimethicone cross polymer) in decamethylcyclopentasiloxane. In some embodiments, the second silicone excipient blend is present from about 5% w/w to about 20% w/w. In some embodiments, the second silicone excipient blend is present from about 6% w/w to about 20% w/w, from about 7% w/w to about 20% w/w, from about 8% w/w to about 20% w/w, from about 9% w/w to about 20% w/w, from about 10% w/w to about 20% w/w, from about 11% w/w to about 20% w/w, from about 12% w/w to about 20% w/w, from about 13% w/w to about 20% w/w, from about 14% w/w to about 20% w/w, from about 15% w/w to about 20% w/w, from about 16% w/w to about 20% w/w, from about 17% w/w to about 20% w/w, from about 18% w/w to about 20% w/w, from about 19% w/w to about 20% w/w, from about 6% w/w to about 19% w/w, from about 7% w/w to about 19% w/w, from about 8% w/w to about 19% w/w, from about 9% w/w to about 19% w/w, from about 10% w/w to about 19% w/w, from about 11% w/w to about 19% w/w, from about 12% w/w to about 19% w/w, from about 13% w/w to about 19% w/w, from about 14% w/w to about 19% w/w, from about 15% w/w to about 19% w/w, from about 16% w/w to about 19% w/w, from about 17% w/w to about 19% w/w, from about 18% w/w to about 19% w/w, from about 6% w/w to about 18% w/w, from about 7% w/w to about 18% w/w, from about 8% w/w to about 18% w/w, from about 9% w/w to about 18% w/w, from about 10% w/w to about 18% w/w, from about 11% w/w to about 18% w/w, from about 12% w/w to about 18% w/w, from about 13% w/w to about 18% w/w, from about 14% w/w to about 18% w/w, from about 15% w/w to about 18% w/w, from about 16% w/w to about 18% w/w, from about 17% w/w to about 18% w/w, from about 6% w/w to about 17% w/w, from about 7% w/w to about 17% w/w, from about 8% w/w to about 17% w/w, from about 9% w/w to about 17% w/w, from about 10% w/w to about 17% w/w, from about 11% w/w to about 17% w/w, from about 12% w/w to about 17% w/w, from about 13% w/w to about 17% w/w, from about 14% w/w to about 17% w/w, from about 15% w/w to about 17% w/w, from about 16% w/w to about 17% w/w, from about 6% w/w to about 16% w/w, from about 7% w/w to about 16% w/w, from about 8% w/w to about 16% w/w, from about 9% w/w to about 16% w/w, from about 10% w/w to about 16% w/w, from about 11% w/w to about 16% w/w, from about 12% w/w to about 16% w/w, from about 13% w/w to about 16% w/w, from about 14% w/w to about 16% w/w, from about 15% w/w to about 16% w/w, from about 6% w/w to about 15% w/w, from about 7% w/w to about 15% w/w, from about 8% w/w to about 15% w/w, from about 9% w/w to about 15% w/w, from about 10% w/w to about 15% w/w, from about 11% w/w to about 15% w/w, from about 12% w/w to about 15% w/w, from about 13% w/w to about 15% w/w, from about 14% w/w to about 15% w/w, from about 6% w/w to about 14% w/w, from about 7% w/w to about 14% w/w, from about 8% w/w to about 14% w/w, from about 9% w/w to about 14% w/w, from about 10% w/w to about 14% w/w, from about 11% w/w to about 14% w/w, from about 12% w/w to about 14% w/w, from about 13% w/w to about 14% w/w, from about 6% w/w to about 13% w/w, from about 7% w/w to about 13% w/w, from about 8% w/w to about 13% w/w, from about 9% w/w to about 13% w/w, from about 10% w/w to about 13% w/w, from about 11% w/w to about 13% w/w, from about 12% w/w to about 13% w/w, from about 6% w/w to about 12% w/w, from about 7% w/w to about 12% w/w, from about 8% w/w to about 12% w/w, from about 9% w/w to about 12% w/w, from about 10% w/w to about 12% w/w, from about 11% w/w to about 12% w/w, from about 6% w/w to about 11% w/w, from about 7% w/w to about 11% w/w, from about 8% w/w to about 11% w/w, from about 9% w/w to about 11% w/w, from about 10% w/w to about 11% w/w, from about 6% w/w to about 10% w/w, from about 7% w/w to about 10% w/w, from about 8% w/w to about 10% w/w, from about 9% w/w to about 10% w/w, from about 6% w/w to about 9% w/w, from about 7% w/w to about 9% w/w, from about 8% w/w to about 9% w/w, from about 6% w/w to about 8% w/w, from about 7% w/w to about 8% w/w, or from about 6% w/w to about 7% w/w. In some embodiments, the second silicone excipient blend is present at about 5, 6, 7, 8, 9, 10, 11, 12, 13, 14, 15, 16, 17, 18, 19 or 20% (w/w). The numerical values above represent amounts of silicone excipient in % (w/w). The second silicone excipient may be present in a quantity sufficient (q.s.) such that the total of all components (i.e. active pharmaceutical ingredients, silicone excipient blends, and lipid excipients) present in a non-aqueous composition equals 100% w/w. For example where the total of active pharmaceutical ingredients, silicone excipient blends, and lipid excipients in a non-aqueous composition is 36.35%, the amount of the second silicone excipient is 63.65% w/w thereby resulting in a total of 100% w/w for all components present in the non-aqueous composition.

In other embodiments, the third silicone excipient blend includes polydimethylcyclosiloxanes. Polydiemthylcyclosiloxanes are cyclic dimethicones including multiple monomer [SiO(CH₃)₂] units. A non-limiting example of a silicone excipient blend including polydimethylcyclosiloxanes is ST-Cyclomethicone®5-NF. ST-Cyclomethicone®5-NF is a clear, colorless, volatile polydimethylcyclosiloxane composed mainly of decamethylcyclopentasiloxane. In some embodiments, the third silicone excipient blend is present from about 10% w/w to about 30% w/w. In some embodiments, the third silicone excipient blend is present from about 12% w/w to about 30% w/w, from about 14% w/w to about 30% w/w, from about 16% w/w to about 30% w/w, from about 18% w/w to about 30% w/w, from about 20% w/w to about 30% w/w, from about 22% w/w to about 30% w/w, from about 24% w/w to about 30% w/w, from about 26% w/w to about 30% w/w, from about 28% w/w to about 30% w/w, from about 12% w/w to about 28% w/w, from about 14% w/w to about 28% w/w, from about 16% w/w to about 28% w/w, from about 18% w/w to about 28% w/w, from about 20% w/w to about 28% w/w, from about 22% w/w to about 28% w/w, from about 24% w/w to about 28% w/w, from about 26% w/w to about 28% w/w, from about 12% w/w to about 26% w/w, from about 14% w/w to about 26% w/w, from about 16% w/w to about 26% w/w, from about 18% w/w to about 26% w/w, from about 20% w/w to about 26% w/w, from about 22% w/w to about 26% w/w, from about 24% w/w to about 26% w/w, from about 12% w/w to about 24% w/w, from about 14% w/w to about 24% w/w, from about 16% w/w to about 24% w/w, from about 18% w/w to about 24% w/w, from about 20% w/w to about 24% w/w, from about 22% w/w to about 24% w/w, from about 12% w/w to about 22% w/w, from about 14% w/w to about 22% w/w, from about 16% w/w to about 22% w/w, from about 18% w/w to about 22% w/w, from about 20% w/w to about 22% w/w, from about 12% w/w to about 20% w/w, from about 14% w/w to about 20% w/w, from about 16% w/w to about 20% w/w, from about 18% w/w to about 20% w/w, from about 12% w/w to about 18% w/w, from about 14% w/w to about 18% w/w, from about 16% w/w to about 18% w/w, from about 12% w/w to about 16% w/w, from about 14% w/w to about 16% w/w, or from about 12% w/w to about 14% w/w. In some embodiments, the third silicone excipient blend is present at about 10, 11, 12, 13, 14, 15, 16, 17, 18, 19, 20, 21, 22, 23, 24, 25, 26, 27, 28, 29 or 30% (w/w). The numerical values above represent amounts of silicone excipient in % (w/w). The third silicone excipient may be present in a quantity sufficient (q.s.) such that the total of all components (i.e. active pharmaceutical ingredients, silicone excipient blends, and lipid excipients) present in a non-aqueous composition equals 100% w/w. For example where the total of active pharmaceutical ingredients, silicone excipient blends, and lipid excipients in a non-aqueous composition is 36.35%, the amount of the third silicone excipient is 63.65% w/w thereby resulting in a total of 100% w/w for all components present in the non-aqueous composition.

The silicone excipient blend according to the embodiments provided herein may include a silicone compound and an acceptable silicone excipient blend carrier. Where the silicone excipient blend includes a silicone compound and an acceptable silicone excipient blend carrier, the silicone compound is combined with an agent which is not a silicone compound. Examples for acceptable silicone excipient blend carriers are stearyl alcohol, isostearyl alcohol, and 1-dodecene. Thus, a silicone excipient blend as provided herein may include one silicone compound. In some embodiments, the fourth silicone excipient blend includes alkylmethyl siloxane copolyol, isostearyl alcohol and 1-dodecene. Alkylmethyl siloxane copolyol is a branched dimethiconol modified with alkyl and polyether groups also known as lauryl PEG-9 polydimethylsiloxyethyl dimethicone. A non-limiting example of a silicone excipient blend including alkylmethyl siloxane copolyol is Emulsifier®10. Emulsifier®10 is a mixture of alkylmethyl siloxane copolyol, isostearyl alcohol and 1-dodecene. In some embodiments, the fourth silicone excipient blend is present from about 0.5% w/w to about 5% w/w. In some embodiments, the fourth silicone excipient blend is present from about 1% w/w to about 5% w/w, from about 1.5% w/w to about 5% w/w, from about 2% w/w to about 5% w/w, from about 2.5% w/w to about 5% w/w, from about 3% w/w to about 5% w/w, from about 3.5% w/w to about 5% w/w, from about 4% w/w to about 5% w/w, from about 4.5% w/w to about 5% w/w, from about 1% w/w to about 4.5% w/w, from about 1.5% w/w to about 4.5% w/w, from about 2% w/w to about 4.5% w/w, from about 2.5% w/w to about 4.5% w/w, from about 3% w/w to about 4.5% w/w, from about 3.5% w/w to about 4.5% w/w, from about 4% w/w to about 4.5% w/w, from about 1% w/w to about 4% w/w, from about 1.5% w/w to about 4% w/w, from about 2% w/w to about 4% w/w, from about 2.5% w/w to about 4% w/w, from about 3% w/w to about 4% w/w, from about 3.5% w/w to about 4% w/w, from about 1% w/w to about 3.5% w/w, from about 1.5% w/w to about 3.5% w/w, from about 2% w/w to about 3.5% w/w, from about 2.5% w/w to about 3.5% w/w, from about 3% w/w to about 3.5% w/w, from about 1% w/w to about 3% w/w, from about 1.5% w/w to about 3% w/w, from about 2% w/w to about 3% w/w, from about 2.5% w/w to about 3% w/w, from about 1% w/w to about 2.5% w/w, from about 1.5% w/w to about 2.5% w/w, from about 2% w/w to about 2.5% w/w, from about 1% w/w to about 2% w/w, from about 1.5% w/w to about 2% w/w, or from about 1% w/w to about 1.5% w/w. In some embodiments, the fourth silicone excipient blend is present at about 0.5, 1, 1.5, 2, 2.5, 3, 3.5, 4, 4.5 or 5% (w/w). The numerical values above represent amounts of silicone excipient in % (w/w).

In some embodiments, the fifth silicone excipient blend includes stearyloxytrimethylsilane and stearyl alcohol. Stearyloxytrimethylsilane refers, in the customary sense, to CAS Registry No. 18748-98-6 and stearyl alcohol refers, in the customary sense, to CAS Registry Number No. 112-92-5. A non-limiting example of a silicone excipient blend including stearyloxytrimethylsilane and stearyl alcohol is Silky Wax®10. Silky Wax®10 is a soft, solid mixture of stearyloxytrimethylsilane and stearyl alcohol. In some embodiments, the fifth silicone excipient blend is present from about 5% w/w to about 15% w/w. In some embodiments, the fifth silicone excipient blend is present from about 6% w/w to about 15% w/w, from about 7% w/w to about 15% w/w, from about 8% w/w to about 15% w/w, from about 9% w/w to about 15% w/w, from about 10% w/w to about 15% w/w, from about 11% w/w to about 15% w/w, from about 12% w/w to about 15% w/w, from about 13% w/w to about 15% w/w, from about 14% w/w to about 15% w/w, from about 6% w/w to about 14% w/w, from about 7% w/w to about 14% w/w, from about 8% w/w to about 14% w/w, from about 9% w/w to about 14% w/w, from about 10% w/w to about 14% w/w, from about 11% w/w to about 14% w/w, from about 12% w/w to about 14% w/w, from about 13% w/w to about 14% w/w, from about 6% w/w to about 13% w/w, from about 7% w/w to about 13% w/w, from about 8% w/w to about 13% w/w, from about 9% w/w to about 13% w/w, from about 10% w/w to about 13% w/w, from about 11% w/w to about 13% w/w, from about 12% w/w to about 13% w/w, from about 6% w/w to about 12% w/w, from about 7% w/w to about 12% w/w, from about 8% w/w to about 12% w/w, from about 9% w/w to about 12% w/w, from about 10% w/w to about 12% w/w, from about 11% w/w to about 12% w/w, from about 6% w/w to about 11% w/w, from about 7% w/w to about 11% w/w, from about 8% w/w to about 11% w/w, from about 9% w/w to about 11% w/w, from about 10% w/w to about 11% w/w, from about 6% w/w to about 10% w/w, from about 7% w/w to about 10% w/w, from about 8% w/w to about 10% w/w, from about 9% w/w to about 10% w/w, from about 6% w/w to about 9% w/w, from about 7% w/w to about 9% w/w, from about 8% w/w to about 9% w/w, from about 6% w/w to about 8% w/w, from about 7% w/w to about 8% w/w, or from about 6% w/w to about 7% w/w. In some embodiments, the fifth silicone excipient blend is present at about 5, 6, 7, 8, 9, 10, 11, 12, 13, 14, or 15% (w/w). The numerical values above represent amounts of silicone excipient in % (w/w).

In other embodiments, the sixth silicone excipient blend includes dimethiconol and hexamethyldisiloxane. Dimethiconol refers, in the customary sense, to CAS Registry No. 70131-67 and hexamethyldisiloxane, in the customary sense, to CAS Registry Number No. 107-46-0. A non-limiting example of a silicone excipient blend including is dimethiconol in hexamethyldisiloxane Silmogen Carrier®. Silmogen Carrier® is a blend of approximately 1% of an ultra high viscosity dimethiconol in a volatile silicone fluid (hexamethyl-disiloxane). In some embodiments, the sixth silicone excipient blend is present from about 5% w/w to about 10% w/w. In some embodiments, the sixth silicone excipient blend is present from about 5.5% w/w to about 10% w/w, from about 6% w/w to about 10% w/w, from about 6.5% w/w to about 10% w/w, from about 7% w/w to about 10% w/w, from about 7.5% w/w to about 10% w/w, from about 8% w/w to about 10% w/w, from about 8.5% w/w to about 10% w/w, from about 9% w/w to about 10% w/w, from about 9.5% w/w to about 10% w/w, from about 5% w/w to about 9.5% w/w, 5.5% w/w to about 9.5% w/w, from about 6% w/w to about 9.5% w/w, from about 6.5% w/w to about 9.5% w/w, from about 7% w/w to about 9.5% w/w, from about 7.5% w/w to about 9.5% w/w, from about 8% w/w to about 9.5% w/w, from about 8.5% w/w to about 9.5% w/w, from about 9% w/w to about 9.5% w/w, from about 5% w/w to about 9% w/w, 5.5% w/w to about 9% w/w, from about 6% w/w to about 9% w/w, from about 6.5% w/w to about 9% w/w, from about 7% w/w to about 9% w/w, from about 7.5% w/w to about 9% w/w, from about 8% w/w to about 9% w/w, from about 8.5% w/w to about 9% w/w, from about 5% w/w to about 8.5% w/w, 5.5% w/w to about 8.5% w/w, from about 6% w/w to about 8.5% w/w, from about 6.5% w/w to about 8.5% w/w, from about 7% w/w to about 8.5% w/w, from about 7.5% w/w to about 8.5% w/w, from about 8% w/w to about 8.5% w/w, from about 5% w/w to about 8% w/w, 5.5% w/w to about 8% w/w, from about 6% w/w to about 8% w/w, from about 6.5% w/w to about 8% w/w, from about 7% w/w to about 8% w/w, from about 7.5% w/w to about 8% w/w, from about 5% w/w to about 7.5% w/w, 5.5% w/w to about 7.5% w/w, from about 6% w/w to about 7.5% w/w, from about 6.5% w/w to about 7.5% w/w, from about 7% w/w to about 7.5% w/w, from about 5% w/w to about 7% w/w, 5.5% w/w to about 7% w/w, from about 6% w/w to about 7% w/w, from about 6.5% w/w to about 7% w/w, from about 5% w/w to about 6.5% w/w, 5.5% w/w to about 6.5% w/w, or from about 6% w/w to about 6.5% w/w. The numerical values above represent amounts of silicone excipient in % (w/w). In some embodiments, the sixth silicone excipient blend is present at about 5, 5.5, 6, 6.5, 7, 7.5, 8, 8.5, 9, 9.5 or 10% (w/w). The sixth silicone excipient may be present in a quantity sufficient (q.s.) that the total of all components (i.e. active pharmaceutical ingredients, silicone excipient blends, and lipid excipients) present in a non-aqueous composition equals 100% w/w. For example where the total of active pharmaceutical ingredients, silicone excipient blends, and lipid excipients in a non-aqueous composition is 36.35%, the amount of the sixth silicone excipient is 63.65% w/w thereby resulting in a total of 100% w/w for all components present in the non-aqueous composition.

In other embodiments, the seventh silicone excipient blend includes alkylmethyl siloxane wax. Alkylmethyl siloxane wax refers to a C₃₀₋₄₅ alkyl methicone. A non-limiting example of a silicone excipient blend including alkylmethyl siloxane wax is ST-Wax®30. ST-Wax®30 is an occlusive siloxane wax which can be used t replace occlusive organic excipients in ointments, emulsions or stick formulations. In some embodiments, the seventh silicone excipient blend is present from about 5% w/w to about 15% w/w. In some embodiments, the seventh silicone excipient blend is present from about 6% w/w to about 15% w/w, from about 7% w/w to about 15% w/w, from about 8% w/w to about 15% w/w, from about 9% w/w to about 15% w/w, from about 10% w/w to about 15% w/w, from about 11% w/w to about 15% w/w, from about 12% w/w to about 15% w/w, from about 13% w/w to about 15% w/w, from about 14% w/w to about 15% w/w, from about 6% w/w to about 14% w/w, from about 7% w/w to about 14% w/w, from about 8% w/w to about 14% w/w, from about 9% w/w to about 14% w/w, from about 10% w/w to about 14% w/w, from about 11% w/w to about 14% w/w, from about 12% w/w to about 14% w/w, from about 13% w/w to about 14% w/w, from about 6% w/w to about 13% w/w, from about 7% w/w to about 13% w/w, from about 8% w/w to about 13% w/w, from about 9% w/w to about 13% w/w, from about 10% w/w to about 13% w/w, from about 11% w/w to about 13% w/w, from about 12% w/w to about 13% w/w, from about 6% w/w to about 12% w/w, from about 7% w/w to about 12% w/w, from about 8% w/w to about 12% w/w, from about 9% w/w to about 12% w/w, from about 10% w/w to about 12% w/w, from about 11% w/w to about 12% w/w, from about 6% w/w to about 11% w/w, from about 7% w/w to about 11% w/w, from about 8% w/w to about 11% w/w, from about 9% w/w to about 11% w/w, from about 10% w/w to about 11% w/w, from about 6% w/w to about 10% w/w, from about 7% w/w to about 10% w/w, from about 8% w/w to about 10% w/w, from about 9% w/w to about 10% w/w, from about 6% w/w to about 9% w/w, from about 7% w/w to about 9% w/w, from about 8% w/w to about 9% w/w, from about 6% w/w to about 8% w/w, from about 7% w/w to about 8% w/w, or from about 6% w/w to about 7% w/w. In some embodiments, the seventh silicone excipient blend is present at about 5, 6, 7, 8, 9, 10, 11, 12, 13, 14, or 15% (w/w). The numerical values above represent amounts of silicone excipient in % (w/w).

Table 1, 2 and 3 describe various examples of combinations of effective amounts of silicone excipient blends useful in the methods, products and compositions provided herein. In particular, Table 1 provides 156 different combinations of concentrations of Elastomer®10, as shown in the first column labeled “Elastomer®10”, and Dimethiconol Blend®20, as shown in the first row labeled “Dimethiconol Blend®20.” Specific concentrations of Elastomer®10 and Dimethiconol Blend®20 for each of the combinations described in Table 1 and numbered from 1 to 156 are shown, respectively, in the cells in the first column and in the first row, which correspond to the numbered cell.

Table 2 provides 335 different combinations of concentrations of Cyclomethicone, as shown in the first column labeled “Cyclomethicone”, and Elastomer®10, as shown in the first row labeled “Elastomer®10.” Specific concentrations of Cyclomethicone and Elastomer®10 for each of the combinations described in Table 2 and numbered from 157 to 492 are shown, respectively, in the cells in the first column and in the first row, which correspond to the numbered cell.

Table 3 provides 109 different combinations of concentrations of ST-Wax®30, as shown in the first column labeled “ST-Wax®30”, and Dimethiconol Blend®20, as shown in the first row labeled “Dimethiconol Blend®20.” Specific concentrations of ST-Wax®30 and Dimethiconol Blend®20 for each of the combinations described in Table 3 and numbered from 493 to 602 are shown, respectively, in the cells in the first column and in the first row, which correspond to the numbered cell.

The compositions and products provided herein include combinations of Elastomer®10, Dimethiconol Blend®20, Cyclomethicone, and ST-Wax®30, respectively. Each of the 156 combinations of concentrations in Table 1 may be combined with any of the 335 combinations of concentrations of Table 2 and/or any of the 109 combinations of Table 3, resulting in 52,260 (combination of concentrations in Table 1 and 2), 17,004 (combination of concentrations in Table 1 and 3) or 5,696340 (combination of concentrations of Table 1, 2, and 3) possible combinations of concentrations. Therefore, 5,696340 individual combination products of Elastomer®10, Dimethiconol Blend®20, Cyclomethicone, and ST-Wax®30 are specifically disclosed herein and are useful in the compositions, products and methods provided herein.

TABLE 1
Effective Amounts of Elastomer ® 10 and Dimethiconol Blend ® 20

| Elastomer ® 10 | Dimethiconol Blend ® 20 w/w | | | | | | | | | |
|---|---|---|---|---|---|---|---|---|---|---|
| w/w | 1% | 2.00% | 3% | 4.00% | 5% | 6.00% | 7% | 8.00% | 9% | 10.00% |
| 5% | 1 | 13 | 29 | 45 | 61 | 77 | 93 | 109 | 125 | 141 |
| 6.00% | 2 | 14 | 30 | 46 | 62 | 78 | 94 | 110 | 126 | 142 |
| 7% | 3 | 15 | 31 | 47 | 63 | 79 | 95 | 111 | 127 | 143 |
| 8.00% | 4 | 16 | 32 | 48 | 64 | 80 | 96 | 112 | 128 | 144 |
| 9% | 5 | 17 | 33 | 49 | 65 | 81 | 97 | 113 | 129 | 145 |
| 10.00% | 6 | 18 | 34 | 50 | 66 | 82 | 98 | 114 | 130 | 146 |
| 11% | 7 | 19 | 35 | 51 | 67 | 83 | 99 | 115 | 131 | 147 |
| 12.00% | 8 | 20 | 36 | 52 | 68 | 84 | 100 | 116 | 132 | 148 |
| 13% | 9 | 21 | 37 | 53 | 69 | 85 | 101 | 117 | 133 | 149 |
| 14.00% | 10 | 22 | 38 | 54 | 70 | 86 | 102 | 118 | 134 | 150 |
| 15% | 11 | 23 | 39 | 55 | 71 | 87 | 103 | 119 | 135 | 151 |
| 16.00% | 12 | 24 | 40 | 56 | 72 | 88 | 104 | 120 | 136 | 152 |
| 17% | 13 | 25 | 41 | 57 | 73 | 89 | 105 | 121 | 137 | 153 |
| 18.00% | 14 | 26 | 42 | 58 | 74 | 90 | 106 | 122 | 138 | 154 |
| 19% | 15 | 27 | 43 | 59 | 75 | 91 | 107 | 123 | 139 | 155 |
| 20.00% | 16 | 28 | 44 | 60 | 76 | 92 | 108 | 124 | 140 | 156 |

TABLE 2
Effective Amounts of Cyclomethicone and Elastomer ® 10

| Cyclo- | | | | | | | | | | | | | | | | |
|---|---|---|---|---|---|---|---|---|---|---|---|---|---|---|---|---|
| methicone | Elastomer ® 10 w/w | | | | | | | | | | | | | | | |
| w/w | 5% | 6.00% | 7% | 8.00% | 9% | 10.00% | 11% | 12.00% | 13% | 14.00% | 15% | 16.00% | 17% | 18.00% | 19% | 20.00% |
| 10% | 157 | 178 | 199 | 220 | 241 | 262 | 283 | 304 | 325 | 346 | 367 | 388 | 409 | 430 | 451 | 472 |
| 11.00% | 158 | 179 | 200 | 221 | 242 | 263 | 284 | 305 | 326 | 347 | 368 | 389 | 410 | 431 | 452 | 473 |
| 12% | 159 | 180 | 201 | 222 | 243 | 264 | 285 | 306 | 327 | 348 | 369 | 390 | 411 | 432 | 453 | 474 |
| 13.00% | 160 | 181 | 202 | 223 | 244 | 265 | 286 | 307 | 328 | 349 | 370 | 391 | 412 | 433 | 454 | 475 |
| 14% | 161 | 182 | 203 | 224 | 245 | 266 | 287 | 308 | 329 | 350 | 371 | 392 | 413 | 434 | 455 | 476 |
| 15.00% | 162 | 183 | 204 | 225 | 246 | 267 | 288 | 309 | 330 | 351 | 372 | 393 | 414 | 435 | 456 | 477 |
| 16% | 163 | 184 | 205 | 226 | 247 | 268 | 289 | 310 | 331 | 352 | 373 | 394 | 415 | 436 | 457 | 478 |
| 17.00% | 164 | 185 | 206 | 227 | 248 | 269 | 290 | 311 | 332 | 353 | 374 | 395 | 416 | 437 | 458 | 479 |
| 18% | 165 | 186 | 207 | 228 | 249 | 270 | 291 | 312 | 333 | 354 | 375 | 396 | 417 | 438 | 459 | 480 |
| 19.00% | 166 | 187 | 208 | 229 | 250 | 271 | 292 | 313 | 334 | 355 | 376 | 397 | 418 | 439 | 460 | 481 |
| 20% | 167 | 188 | 209 | 230 | 251 | 272 | 293 | 314 | 335 | 356 | 377 | 398 | 419 | 440 | 461 | 482 |
| 21.00% | 168 | 189 | 210 | 231 | 252 | 273 | 294 | 315 | 336 | 357 | 378 | 399 | 420 | 441 | 462 | 483 |
| 22% | 169 | 190 | 211 | 232 | 253 | 274 | 295 | 316 | 337 | 358 | 379 | 400 | 421 | 442 | 463 | 484 |
| 23.00% | 170 | 191 | 212 | 233 | 254 | 275 | 296 | 317 | 338 | 359 | 380 | 401 | 422 | 443 | 464 | 485 |
| 24% | 171 | 192 | 213 | 234 | 255 | 276 | 297 | 318 | 339 | 360 | 381 | 402 | 423 | 444 | 465 | 486 |
| 25.00% | 172 | 193 | 214 | 235 | 256 | 277 | 298 | 319 | 340 | 361 | 382 | 403 | 424 | 445 | 466 | 487 |
| 26% | 173 | 194 | 215 | 236 | 257 | 278 | 299 | 320 | 341 | 362 | 383 | 404 | 425 | 446 | 467 | 488 |
| 27.00% | 174 | 195 | 216 | 237 | 258 | 279 | 300 | 321 | 342 | 363 | 384 | 405 | 426 | 447 | 468 | 489 |
| 28% | 175 | 196 | 217 | 238 | 259 | 280 | 301 | 322 | 343 | 364 | 385 | 406 | 427 | 448 | 469 | 490 |
| 29.00% | 176 | 197 | 218 | 239 | 260 | 281 | 302 | 323 | 344 | 365 | 386 | 407 | 428 | 449 | 470 | 491 |
| 30% | 177 | 198 | 219 | 240 | 261 | 282 | 303 | 324 | 345 | 366 | 387 | 408 | 429 | 450 | 471 | 492 |

TABLE 3
Effective Amounts of ST Wax ® 30 and Dimethiconol Blend ® 20

| ST Wax ® 30 | Dimethiconol Blend ® 20 w/w | | | | | | | | | |
|---|---|---|---|---|---|---|---|---|---|---|
| w/w | 1% | 2.00% | 3% | 4.00% | 5% | 6.00% | 7% | 8.00% | 9% | 10.00% |
| 5% | 493 | 504 | 515 | 526 | 537 | 548 | 559 | 570 | 581 | 592 |
| 6.00% | 494 | 505 | 516 | 527 | 538 | 549 | 560 | 571 | 582 | 593 |
| 7% | 495 | 506 | 517 | 528 | 539 | 550 | 561 | 572 | 583 | 594 |
| 8.00% | 496 | 507 | 518 | 529 | 540 | 551 | 562 | 573 | 584 | 595 |
| 9% | 497 | 508 | 519 | 530 | 541 | 552 | 563 | 574 | 585 | 596 |
| 10.00% | 498 | 509 | 520 | 531 | 542 | 553 | 564 | 575 | 586 | 597 |
| 11% | 499 | 510 | 521 | 532 | 543 | 554 | 565 | 576 | 587 | 598 |
| 12.00% | 500 | 511 | 522 | 533 | 544 | 555 | 566 | 577 | 588 | 599 |
| 13% | 501 | 512 | 523 | 534 | 545 | 556 | 567 | 578 | 589 | 600 |
| 14.00% | 502 | 513 | 524 | 535 | 546 | 557 | 568 | 579 | 590 | 601 |
| 15% | 503 | 514 | 525 | 536 | 547 | 558 | 569 | 580 | 591 | 602 |

The non-aqueous compositions and products according to the embodiments of the present invention may include a lipid excipient or a thickening agent. Thus, in some embodiments, the composition further includes a lipid excipient or a thickening agent. In other embodiments, the composition includes a lipid excipient and a thickening agent. The non-aqueous compositions as provided herein may include at least one lipid excipient. Thus, in some embodiments, the composition includes a plurality of lipid excipients. In some embodiments, the composition includes a plurality of lipid excipients or a thickening agent. In other embodiments, the composition includes a plurality of lipid excipients and a thickening agent. Where the no-aqueous composition includes a plurality of lipid excipients it includes more than one lipid excipient.

The term “lipid excipient” as used herein refers to a lipid-based material that is co-formulated with a pharmaceutical composition. Non-limiting examples include castor oil, linoleic acid, bisabolol, squalane, propylene glycol, isostearyl isostearate, isopropyl myristate, diethylene glycol, dipropylene glycol, mineral oil, vegetable oil, almond oil, petrolatum, microcrystalline wax, lanolin, beeswax, caprylic/capric triglycerides, cetyl alcohol, mineral oil, jojoba seed oil, stearyl alcohol, arachidyl alcohol, behenyl alcohol, and long chain fatty acids (C₁₂-C₂₂). In some embodiments, the lipid excipient is mineral oil. In some further embodiments, the mineral oil is present from about 0.5% w/w to about 10% w/w. In other embodiments, the lipid excipient is capric/caprylic triglyceride. In some further embodiments, the capric/caprylic triglyceride is present from about 5% w/w to about 15% w/w. In some embodiments, the lipid excipient is beeswax. In some further embodiments, the beeswax is present from about 10% w/w to about 30% w/w. In some embodiments, the lipid excipient is lanolin. In some further embodiments, the lanolin is present from about 5% w/w to about 10% w/w. In some embodiments, the lipid excipient is cetyl alcohol. In some further embodiments, the cetyl alcohol is present from about 5% w/w to about 10% w/w. In some embodiments, the lipid excipient is castor oil. In some embodiments, the lipid excipient is isopropyl myristate. In some further embodiments, the isopropyl myristate is present from about 0.5% w/w to about 15% w/w. In some embodiments, the lipid excipient is petrolatum. Petrolatum refers, in the customary sense, to CAS Registry No. 8009-03-8. Petrolatum is a semi-solid mixture of hydrocarbons (with carbon numbers mainly higher than 25). In some embodiments, the lipid excipient is vegetable oil. In some further embodiments, the vegetable oil is present from about 0.5% w/w to about 5% w/w. In some embodiments, the lipid excipient is almond oil. In some further embodiments, the almond oil is present from about 0.5% w/w to about 5% w/w.

The formulation's viscosity is a factor that determines how well the formulation sticks to the skin or ophthalmic tissue or does not run off the skin or ophthalmic tissue when applied. The viscosity of the formulation can be optimized using one or more pharmaceutically acceptable thickening agents that do not significantly interact with the components of the formulation, do not significantly reduce flux of the formulation, and do not cause stinging or irritation. Non-limiting examples of suitable thickeners useful herein include cellulosic polymers, such as gum arabic, gum acacia, gum tragacanth, locust bean gum, guar gum, hydroxypropyl guar, xanthan gum, talc, cellulose gum, sclerotium gum, carageenan gum, karaya gum, cellulose gum, rosin, methylcellulose, hydroxyethylcellulose, hydroxypropylcellulose, hydroxymethylcellulosf, hydroxypropylmethylcellulose, methylhydroxyethylcellulose, cetyl hydroxyethylcellulose, carboxymethylcellulose, corn starch, hydroxypropyl starch phosphate, distarch phosphate, distarch dimethylene urea, aluminum starch octenyl succinate, maltodextrin, dextran, poly(acrylamide), PEG-150 distearate, PEG-150/decyl alcohol/SMDI copolymer, PEG-150/stearyl alcohol/SMDI copolymer, PEG-180/Laureth-50/TMMG copolymer, Polyether 1, acrylic acid/acrylamidomethyl propane sulfonic acid copolymer, acrylate/C10-30 alkyl acrylate cross polymer, acrylate/beheneth-25 methacrylate copolymer, acrylate/steareth-20 methacrylate copolymer, acrylate/steareth-20 copolymer, acrylate/VA cross polymer, acrylic acid/acrylonitrogen copolymer, ammonium acryloyldimethyltaurate/beheneth-25 methacrylate copolymer, ammonium acryloyldimethyltaurate/VP copolymer, sodium acrylate copolymer, PVM/MA decadiene cross polymer, alginic acid, propylene glycol alginate, dimethicone, silica dimethyl silylate, a dimethylacrylamide/acrylic acid/polystyrene ethyl methacrylate copolymer, derivatives thereof, and mixtures thereof. In some embodiments, the thickening agent is a talc. In some further embodiments, the talc is present from about 2% w/w to about 5% w/w.

Other ingredients, which may optionally be included into the topical non-aqueous compositions and products according to embodiments of the present invention, include humectants, such as propylene glycol; solvents, such as alcohols, sun filters, such as titanium dioxide, zinc oxide, and calcium carbonate; and anti-microbial preservatives, such as methylparaben and propylparaben. An organic or inorganic base may also be included, such as sodium hydroxide, which is used to adjust the pH of the initial components and the final product. Generally, ophthalmically acceptable excipients commonly known in the fields of ophthalmology and cosmetology as useful in topical compositions, and any non-toxic, inert, and effective topical carriers, are contemplated as useful in the compositions and products according to the embodiments of the present invention.

As described above, the non-aqueous compositions provided herein include an active pharmaceutical ingredient. In some embodiments, the non-aqueous composition includes cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, or gatifloxacin. The effective amounts for each of the individual active pharmaceutical ingredient (e.g. cyclosporine, tacrolimus, phentolamine) are described herein. For example, cyclosporine may be present in an amount approximately equal to or less than about 0.4% w/w, tacrolimus may be present in an amount approximately equal to or less than about 0.1% w/w, phentolamine may be present in an amount approximately equal to or less than about 1% w/w, testosterone may be present in an amount approximately equal to or less than about 5% w/w, dihydrotestosterone may be present in an amount approximately equal to or less than about 5% w/w and testosterone propionate may be present in an amount approximately equal to or less than about 5% w/w. The non-aqueous compositions of the present invention include effective amounts of the active pharmaceutical ingredients as provided herein at the concentrations described for each active pharmaceutical ingredient.

In some embodiments, the non-aqueous composition consists essentially of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, or gatifloxacin, a plurality of lipid excipients, and a silicone excipient. In some embodiments, the non-aqueous composition consists essentially of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, or gatifloxacin, a plurality of lipid excipients; and a plurality of silicone excipients. Where the non-aqueous composition consists essentially of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, or gatifloxacin, a plurality of lipid excipients, and a plurality of silicone excipients, the non-aqueous composition consists of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, or gatifloxacin and any suitable plurality of lipid excipients and silicone excipient or plurality of silicone excipients.

In some embodiments, the non-aqueous composition consists essentially of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, or gatifloxacin, a plurality of lipid excipients, a thickening agent, and a silicone excipient. In some embodiments, the non-aqueous composition consists essentially of cyclosporine, tacrolimus, phentolamine, testosterone, dihydrotestosterone, testosterone propionate, dexamethasone, prednisolone, an EP2 receptor agonist, brimonidine, pilocarpine, a prostaglandin analog, ketorolac, timolol, or gatifloxacin, a plurality of lipid excipients, a thickening agent, and a plurality of silicone excipients.

The ophthalmic pharmaceutical non-aqueous compositions provided herein may be administered in various ways e.g. an emulsion, a foam, a gel, a cream, jelly, solution, suspension, a spray (e.g., a solution), an ointment, ointment films, occlusive films, sustained release films, fast drying films, slow drying films, patches, semi solids or stick formulation comprising a semi-solid vehicle with a melting point near physiological temperature. Topical compositions and products according to embodiments of the present invention can also be formulated as ointments, which are oleaginous and contain little if any water.

In some embodiments, the ophthalmic pharmaceutical formulation is an ointment formulation. Where the ophthalmic pharmaceutical formulation is an ointment formulation, the active pharmaceutical ingredient may be a cyclosporine, tacrolimus, phentolamine, testosterone, dihydrotestosteron, testosterone propionate, dexamethasone, prednisolone, an EP2 receptor agonist, brimonidine, pilocarpine, a prostaglandin analog, ketorolac, timolol, or gatifloxacin. Further, where the ophthalmic pharmaceutical formulation is an ointment formulation, the formulation may include a first silicone excipient blend and a second silicone excipient blend. Thus, in some further embodiments, the silicone excipient is a first silicone excipient blend or a second silicone excipient blend. In some embodiments, the first silicone excipient blend is a mixture of dimethicone and dimethiconol and the second silicone excipient blend is a mixture of alkylmethyl siloxane wax. In other embodiments, the first silicone excipient blend is a mixture of cyclopentasiloxane and dimethicone cross polymer, and the second silicone excipient blend is a mixture of polydimethylcyclopentasiloxanes. In other embodiments, the non-aqueous composition further includes a lipid excipient. In some embodiments, the lipid excipient is petrolatum.

In some embodiments, the ophthalmic pharmaceutical formulation is a gel formulation. Where the ophthalmic pharmaceutical formulation is a gel formulation, the active pharmaceutical ingredient may be a cyclosporine, tacrolimus, phentolamine, testosterone, dihydrotestosteron, testosterone propionate, dexamethasone, prednisolone, an EP2 receptor agonist, brimonidine, pilocarpine, a prostaglandin analog, ketorolac, timolol, or gatifloxacin. Further, where the ophthalmic pharmaceutical formulation is a gel formulation the formulation may include a first silicone excipient blend and a second silicone excipient blend. Thus, in some further embodiments, the silicone excipient is a first silicone excipient blend or a second silicone excipient blend. In some embodiments, the first silicone excipient blend is a mixture of cyclopentasiloxane and dimethicone cross polymer and the second silicone excipient blend is a mixture of polydimethylcyclosiloxanes. In a further embodiment, the non-aqueous composition includes a lipid excipient. In some embodiments, the lipid excipient is isopropyl myristate.

In some embodiments, the ophthalmic pharmaceutical formulation is a spray formulation. Where the ophthalmic pharmaceutical formulation is a spray formulation, the active pharmaceutical ingredient may be a cyclosporine, tacrolimus, phentolamine, testosterone, dihydrotestosteron, testosterone propionate, dexamethasone, prednisolone, an EP2 receptor agonist, brimonidine, pilocarpine, a prostaglandin analog, ketorolac, timolol, or gatifloxacin. Further, where the ophthalmic pharmaceutical formulation is a spray formulation, the formulation may include a mixture of dimethiconol and hexamethyldisiloxane. Thus, in some further embodiments, the silicone excipient is a mixture of dimethiconol and hexamethyldisiloxane. In some further embodiments, the non-aqueous composition includes a thickening agent. In some further embodiments, the thickening agent is talc. Where the ophthalmic pharmaceutical formulation is a spray formulation the formulation may include a first silicone excipient blend and a second silicone excipient blend. Thus, in some embodiments, the silicone excipient is a first silicone excipient blend and a second silicone excipient blend. In some further embodiments, the first silicone excipient blend is a mixture of cyclopentasiloxane and dimethicone cross polymer and the second silicone excipient blend is a mixture of dimethiconol and hexamethyldisiloxane. In some other embodiments, the formulation includes a first silicone excipient blend, a second silicone excipient blend and a third silicone excipient blend. In some further embodiments, the first silicone excipient blend is a mixture of dimethicone and dimethiconol, the second silicone excipient blend is a mixture of cyclopentasiloxane and dimethicone cross polymer, and the third silicone excipient blend is a mixture of polydimethylcyclosiloxanes.

In some embodiments, the ophthalmic pharmaceutical formulation is a stick formulation. Where the ophthalmic pharmaceutical formulation is a stick formulation, the active pharmaceutical ingredient may be a cyclosporine, tacrolimus, phentolamine, testosterone, dihydrotestosteron, testosterone propionate, dexamethasone, prednisolone, an EP2 receptor agonist, brimonidine, pilocarpine, a prostaglandin analog, ketorolac, timolol, or gatifloxacin. Further, where the ophthalmic pharmaceutical formulation is a stick formulation the formulation may include alkylmethyl siloxane wax. Thus, in some further embodiments, the silicone excipient is a alkylmethyl siloxane wax. In some further embodiments, the non-aqueous composition includes a plurality of lipid excipients. Where the ophthalmic pharmaceutical formulation is a stick formulation the formulation may include a first silicone excipient blend and a second silicone excipient blend. Thus, in some embodiments, the silicone excipient is a first silicone excipient blend or a second silicone excipient blend. In some further embodiments, the first silicone excipient blend is a mixture of stearyloxytrimethylsilane and stearyl alcohol, and the second silicone excipient blend is a mixture of polydimethylcyclosiloxanes. In some further embodiments, the non-aqueous composition includes a plurality of lipid excipients.

In some embodiments, the ophthalmic pharmaceutical formulation is an emulsion formulation. Where the ophthalmic pharmaceutical formulation is an emulsion formulation, the active pharmaceutical ingredient may be a cyclosporine, tacrolimus, phentolamine, testosterone, dihydrotestosteron, testosterone propionate, dexamethasone, prednisolone, an EP2 receptor agonist, brimonidine, pilocarpine, a prostaglandin analog, ketorolac, timolol, or gatifloxacin. Further, where the ophthalmic pharmaceutical formulation is an emulsion formulation, the formulation include a mixture of alkylmethyl siloxane copolyol, isostearyl alcohol and 1-dodecene. Thus, in some further embodiments, the silicone excipient is a mixture of alkylmethyl siloxane copolyol, isostearyl alcohol and 1-dodecene. In some further embodiments, the non-aqueous composition includes a lipid excipient. In some further embodiments, the lipid excipient is mineral oil. Where the ophthalmic pharmaceutical formulation is an emulsion formulation the formulation may include a first silicone excipient blend and a second silicone excipient blend. Thus, in some embodiments, the silicone excipient is a first silicone excipient blend or a second silicone excipient blend. In some further embodiments, the first silicone excipient blend is a mixture of alkylmethyl siloxane copolyol, isostearyl alcohol and 1-dodecene, and the second silicone excipient blend is a mixture of dimethicone and dimethiconol. In some further embodiments, the non-aqueous composition of includes a lipid excipient. In some further embodiment, the lipid excipient is vegetable oil. In still a further embodiment, the lipid excipient is almond oil.

### III. Treatment Methods

Methods of treating an ophthalmic disease are provided, including methods of treating glaucoma. Some embodiments of the methods provided herein comprise applying an ophthalmic formulation described herein to the region on or around the eye, which can treat ophthalmic diseases by sustained administration of an effective amount of an active pharmaceutical ingredients and a silicone excipient to the ophthalmic tissue (i.e. conjunctiva, lacrimal tissue or cornea).

In one aspect, a method of treating an ophthalmic disease in a subject in need thereof is provided. The method includes administering to the subject an active pharmaceutical ingredient and a silicone excipient. The active pharmaceutical ingredients useful for the methods according to the embodiments of the present invention are described herein. The active pharmaceutical ingredients include at least one (e.g. one) immunosuppressant (e.g. cyclosporine), at least one (e.g. one) vasodilator agent (e.g. phentolamine), at least one (e.g. one) anti-inflammatory agent (e.g. testosterone), at least one (e.g. one) EP2 receptor agonist (e.g. a compound of Formula Ia), at least one (e.g. one) muscarinic receptor agonist (e.g. pilocarpine), at least one (e.g. one) prostaglandin analog (e.g. bimatoprost), at least one (e.g. one) vasoconstrictor agent (e.g. brimonidine, a compound of Formula (IVa)), or at least one (e.g. one) anti-infective agent (e.g. gatifloxacin).

The methods provided herein include administering a silicone excipient. Silicone excipients suitable for the methods of treating an ophthalmic disease are provided herein and include silicone excipient blends (e.g. a silicone excipient blend including dimethicone and dimethiconol or a cyclopentasiloxane and a dimethicone cross polymer) and combinations thereof. The silicone based excipients provided herein possess unexpectedly advantageous properties in comparison with the conventional ophthalmic excipients, since they are chemically and biologically inert, have low surface tension (i.e. good spreading characteristics o water), improve chemical stability of labile active pharmaceutical ingredients and enable the solubility of hydrophobic active pharmaceutical ingredients.

In some embodiments, the ophthalmic disease is central retinal vein occlusion. In other embodiments, the ophthalmic disease is branch retinal vein occlusion. In other embodiments, the ophthalmic disease is choroidal macular edema. In another embodiment, the ophthalmic disease is diabetic macular edema. In some embodiments, the ophthalmic disease is diabetic macular retinopathy. In other embodiments, the ophthalmic disease is uveitis. In some other embodiments, the ophthalmic disease is age related macular degeneration. In other embodiments, the ophthalmic disease is glaucoma. In some embodiments, the ophthalmic disease is ocular hypertension.

In another aspect, a method of improving vision in a subject in need thereof is provided. The method includes administering to the subject an active pharmaceutical ingredient and a silicone excipient.

### IV. Examples

Embodiments of the present invention are further illustrated by the following examples, which are not to be construed in any way as imposing limitations upon the scope thereof. On the contrary, it is to be clearly understood that resort may be made to various other embodiments, modifications and equivalents, which, after reading the description provided herein, may suggest themselves to those skilled in the art without departing from the spirit of the invention.

### Example 1

Table 4 illustrates an example of an ointment formulation according to the embodiments of the present invention.

TABLE 4

| | | |
|---|---|---|
| Delivery System | Ointment | Ointment |
| Quantity | % w/w | % w/w |

| | Formulation contains |
|---|---|
| | anyone of the below |
| Active Ingredients | active ingredients |
| Cyclosporine | 0.01-0.1 |
| Tacrolimus | 0.01-0.1 |
| Phentolamine | 0.001-1% |
| Testosterone | 0.001-5% |
| Dihydro Testosterone | 0.001-5% |
| Testosterone propionate | 0.001-5% |
| Compound of Formula (Ia) | 0.001-0.1% |
| Compound of Formula (IIa) | 0.0002-0.05% |
| Excipients | |

| | | |
|---|---|---|
| ST-Wax 30 | | 5-15 |
| Dimethiconol Blend 20 | | 5-10 |
| Elastomer 10 | 10-20 | |
| Cyclomethicone 5-NF | 10-20 | |
| Petrolatum | q.s. 100 | q.s. 100 |

### Example 2

Table 5 illustrates an example of a gel formulation according to the embodiments of the present invention.

TABLE 5

| Delivery System | Gel |
|---|---|
| Quantity | % w/w |
| | Formulation contains anyone of |
| Active Ingredients | the below active ingredients |
| Cyclosporine | 0.01-0.1 |
| Tacrolimus | 0.01-0.1 |
| Phentolamine | 0.001-1% |
| Testosterone | 0.001-5% |
| Dihydro Testosterone | 0.001-5% |
| Testosterone propionate | 0.001-5% |
| Compound of Formula (Ia) | 0.001-0.1% |
| Compound of Formula (IIa) | 0.0002-0.05% |
| Excipients | |
| Elastomer 10 | q.s. 100 |
| Cyclomethicone 5-NF | 20-30 |
| Iisopropyimyristate | 0.5-3 |

### Example 3

Table 6 illustrates an example of a spray formulation according to the embodiments of the present invention.

TABLE 6

| Delivery System | Spray | Spray | Spray |
|---|---|---|---|
| Quantity | % w/w | % w/w | % w/w |
| | Formulation | | |
| | contains anyone of the | | |
| Active Ingredients | below active ingredients | | |
| Cyc1osporine | 0.01-0.1 | | |
| Tacrolimus | 0.01-0.1 | | |
| Phentolamine | 0.001-1% | | |
| Testosterone | 0.001-5% | | |
| Dihydro Testosterone | 0.001-5% | | |
| Testosterone propionate | 0.001-5% | | |
| Compound of Formula (Ia) | 0.001-0.1% | | |
| Compound of Formula (IIa) | 0.0002-0.05% | | |
| Excipients | | | |
| Silmogen Carrier | q.s. 100 | q.s. 100 | |
| Talc | 2-5 | | |
| Dimethiconol Blend 20 | | | 1-5 |
| Elastomer 10 | | 5-15 | 5-15 |
| Cyc1omethicone 5-NF | | | q.s. 100 |

### Example 4

Table 7 illustrates an example of a stick formulation according to the embodiments of the present invention.

TABLE 7

| Delivery System | Stick | Stick | Stick |
|---|---|---|---|
| Quantity | % w/w | % w/w | % w/w |
| | Formulation | | |
| | contains anyone of the | | |
| Active Ingredients | below active ingredients | | |
| Cyc1osporine | 0.01-0.1 | | |
| Tacrolimus | 0.01-0.1 | | |
| Phentolamine | 0.001-1% | | |
| Testosterone | 0.001-5% | | |
| Dihydro Testosterone | 0.001-5% | | |
| Testosterone propionate | 0.001-5% | | |
| Compound of | 0.001-0.1% | | |
| Formula (Ia) | | | |
| Compound of | 0.0002-0.05% | | |
| Formula (IIa) | | | |
| Excipients | | | |
| Beeswax | 10-20 | | 10-30 |
| ST-Wax 30 | 8-12 | | 5-10 |
| Lanolin | 5-10 | | |
| Cetyl Alcohol | 5-10 | | |
| Castor Oil | q.s. 100 | | q.s. 100 |
| Jojoba Seed Oil | | | 5-10 |
| Silky Wax 10 | | 5-15 | |
| Cyclomethicone 5-NF | | q.s. 100 | |
| Caprylic/capric triglyceride | | 5-15 | |
| Mineral Oil | | | 5-10 |
| Isopropylmyristate | | 5-15 | |

### Example 5

Table 8 illustrates an example of an emulsion formulation according to the embodiments of the present invention.

TABLE 8

| Delivery System | Emulsion | Emulsion | Emulsion |
|---|---|---|---|
| Quantity | % w/w | % w/w | % w/w |
| | Formulation contains anyone of | | |
| Active Ingredients | the below active ingredients | | |
| Cyclosporine | 0.01-0.1 | | |
| Tacrolimus | 0.01-0.1 | | |
| Phentolamine | 0.001-1% | | |
| Testosterone | 0.001-5% | | |
| Dihydro Testosterone | 0.001-5% | | |
| Testosterone propionate | 0.001-5% | | |
| Compound of Formula (Ia) | 0.001-0.1% | | |
| Compound of Formula (IIa) | 0.0002-0.05% | | |
| Excipients | | | |
| Emulsifier 10 | 0.5-5 | 0.5-5 | 0.5-5 |
| Mineral Oil | | 0.5-5 | |
| Vegetable oil | 0.5-5 | | |
| Almond Oil | | | 0.5-5 |
| Dimethiconol blend 20 | q.s. 100 | | q.s. 100 |

### Example 6

Table 9 illustrate active pharmaceutical ingredients (API) according to the embodiments of the present invention.

TABLE 9

| | | Typical concentration |
|---|---|---|
| | | range in Ophthalmic |
| API | Examples | products |
| Immunosuppressant | Cyclosporine A, | 0.001-0.4% |
| | Cyclosporine analogs | |
| Alpha-adrenergic | Phentolamine | 0.001-2% |
| antagonist | | |
| Steroids | Testosterone, | 0.001-5% |
| | Dexamethasone, | |
| | Prednisolone | |
| EP-2 agonists | Compound of Formula | 0.001-0.1% |
| | (Ia) | |
| | Compound of Formula | 0.0002-0.05% |
| | (IIa) | |
| | Compound of Formula | 0.001-0.1% |
| | (IIIa) | |
| Muscarinics | Pilocarpine | 0.1-6.0% |
| Prostaglandins | Bimatoprost, Latanoprost | 0.001-0.1 |
| Alpha-agonists | Brimonidine, Compound | 0.001-1% |
| | of Formula (IVa); | |
| | Compound of Formula | |
| | (Va); Compound of | |
| | Formula (VI); Compound | |
| | of Formula (VIIa)), | |
| | Compound of Formula | |
| | (VIIIa)) | |
| Antibiotics/anti- | Gatifloxicin | 0.1-1% |
| infectives | | |
| Anti-inflammatory | Ketorolac | 0.01-1% |
| | Steroids | |
| Beta Blockers | Timolol | 0.05-0.5% |

### Example 7

Table 10 illustrates the compositions according to the embodiments provided herein, which were used for in vivo assays.

TABLE 10

| | |
|---|---|
| Component | Concentration (w/w %) |

| | | |
|---|---|---|
| Formulation # | 1 | 2 |
| Compound of | 0.11 | — |
| Formula (VIIIa) | | |
| Compound of | — | 0.1 |
| Formula (IVa) | | |
| Dimethiconol 40 | QS 100% | QS 100% |

In vivo pilot study was conducted using 4-[(S*)-1-(2,3-Dimethyl-phenyl)-ethyl]-1H-imidazole (Compound of Formula (VIIIa)) (0.11% w/w) and 3-[(1S)-1-(1H-imidazol-4-yl)ethyl]-2-methylbenzyl 2-methylpropanoate (Compound of Formula (IVa) (0.1% w/w) in dimethiconol 40. Eight normotensive DB rabbits (pigmented) were divided into 2 groups of 4. Formulations were instilled in the right eye (volume 35 uL). Tonometric measurements conducted at 0, 2, and 4 hours.

What is claimed is:

1. A non-aqueous composition comprising an active pharmaceutical ingredient and a silicone excipient.

2. The non-aqueous composition of claim 1, wherein said active pharmaceutical ingredient is an immunosuppressant, a vasodilator agent, an anti-inflammatory agent, an EP2 receptor agonist, a muscarinic receptor agonist, a prostaglandin analog, a vasoconstrictor agent, or an anti-infective agent.

3. The non-aqueous composition of claim 1, wherein said composition is an ophthalmic pharmaceutical formulation.

4. The non-aqueous composition of claim 1, wherein said active pharmaceutical ingredient is an immunosuppressant.

5. The non-aqueous composition of claim 1, wherein said active pharmaceutical ingredient is a vasodilator agent.

6. The non-aqueous composition of claim 1, wherein said active pharmaceutical ingredient is an anti-inflammatory agent.

7. The non-aqueous composition of claim 1, wherein said active pharmaceutical ingredient is an EP2 receptor agonist.

8. The non-aqueous composition of claim 1, wherein said active pharmaceutical ingredient is a muscarinic receptor agonist.

9. The non-aqueous composition of claim 1, wherein said active pharmaceutical ingredient is a prostaglandin analog.

10. The non-aqueous composition of claim 1, wherein said active pharmaceutical ingredient is a vasoconstrictor agent.

11. The non-aqueous composition of claim 1, wherein said silicone excipient is a first silicone excipient blend, a second silicone excipient blend, a third silicone excipient blend, fourth silicone excipient blend, a fifth silicone excipient blend, a sixth silicone excipient blend or a seventh silicone excipient blend.

12. The non-aqueous composition of claim 11, wherein said first silicone excipient blend comprises a mixture of dimethicone and dimethiconol.

13. The non-aqueous composition of claim 11, wherein said second silicone excipient blend comprises a mixture of cyclopentasiloxane and a dimethicone cross polymer.

14. The non-aqueous composition of claim 11, wherein said third silicone excipient blend comprises a mixture of polydimethylcyclosiloxanes.

15. The non-aqueous composition of claim 11, wherein said fourth silicone excipient blend comprises a mixture of alkylmethyl siloxane copolyol, isostearyl alcohol and 1-dodecene.

16. The non-aqueous composition of claim 11, wherein said fifth silicone excipient blend comprises a mixture of stearyloxytrimethylsilane and stearyl alcohol.

17. The non-aqueous composition of claim 11, wherein said sixth silicone excipient blend comprises a mixture of dimethiconol and hexamethyldisiloxane.

18. The non-aqueous composition of claim 11, wherein said seventh silicone excipient blend comprises alkylmethyl siloxane wax.

19. A method of treating an ophthalmic disease in a subject in need thereof, said method comprising administering to said subject an active pharmaceutical ingredient and a silicone excipient.

20. A method of improving vision in a subject in need thereof, said method comprising administering to said subject an active pharmaceutical ingredient and a silicone excipient.
